(19) United States
(12) Patent Application Publication
(10) Pub. No.: US 2026/0102445 A1
(43) Pub. Date: Apr. 16, 2026

(54) ONCOLYTIC ADENOVIRAL VECTOR AND METHODS OF USE

(71) Applicant: Unleash Immuno Oncolytics, Inc., St. Louis, MO (US)

(72) Inventor: Imre Kovesdi, Rockville, MD (US)

(21) Appl. No.: 19/113,028

(22) PCT Filed: Sep. 20, 2023
(86) PCT No.: PCT/US2023/074623

§ 371 Date: Mar. 19, 2025

Related U.S. Application Data
(60) Provisional application No. 63/408,170, filed on Sep. 20, 2022.

(51) Int. Cl. A61K 35/761; A61K 45/06; A61P 35/00; C07K 14/005; C07K 14/54; C07K 14/705; C12N 7/00

(52) U.S. Cl. CPC A61K 35/761; A61K 45/06; A61P 35/00; C07K 14/005; C07K 14/54; C07K 14/70575; C07K 14/70578; C12N 7/00; C12N 2710/10021; C12N 2710/10022; C12N 2710/10032; C12N 2710/10041

(57) ABSTRACT

Provided herein is a conditionally-replicating serotype 5 adenovirus or adenoviral vector expressing a mutant E1A protein under control of a promoter that is responsive to hypoxia and inflammation and one or more immune modulators under control of a tumor-specific promoter. The adenovirus or adenoviral vector also comprises serotype 3 fiber domain. Also provided is a method of inducing cytotoxicity in tumor cells using a composition containing the adenovirus or adenoviral vector.

### STATEMENT REGARDING RELATED APPLICATIONS

This application claims priority to U.S. Provisional Application No. 63/408,170, filed Sep. 20, 2022, the entire contents of which are incorporated herein by reference for all purposes.

### SEQUENCE LISTING

The text of the computer readable sequence listing filed herewith, titled “UIO-41188-601_SQL”, created Sep. 20, 2023, having a file size of 14,474 bytes, is hereby incorporated by reference in its entirety.

### BACKGROUND

Oncolytic virotherapy (OV) is a promising and exciting new approach for cancer treatment. Oncolytic viruses are genetically engineered or naturally occurring viruses that selectively replicate in and kill cancer cells without harming normal tissues. In addition to the primary effect of cell killing, OVs can also stimulate the immune system. Oncolytic virus immunotherapy involves the use of oncolytic viruses that also activate cells of the immune system, such as dendritic cells and T cells, and represents a promising agent for cancer immunotherapy. Current OV methodologies employ a variety of different viruses, such as adenovirus. Newcastle disease virus, herpes simplex virus, reovirus, parvovirus, and measles virus. One oncolytic virus, talimogene laherparepvec, or T-VEC (IMLYGIC™), has been approved by the FDA for local treatment of unresectable cutaneous, subcutaneous, and nodal lesions in patients with melanoma recurrent after initial surgery. Oncolytic virus immunotherapies are currently being studied in clinical trials for a variety of different cancers, such as bladder, prostate, colorectal, ovarian, lung, breast, and multiple myeloma.

With the rapid evolution of OV, methods for improving the safety and potency of oncolytic viruses are being developed, including, for example, improving tumor selectivity, enhancing infectivity and conditional replication in tumor cells, and maximizing transgene expression for enhanced cytotoxicity or host immune stimulation (see, e.g., Lang et al., J. Clin. Oncol., 36:1419-1427 (2018); Forsyth and Abate-Daga, J. Clin. Oncol., 36:1440-1442 (2018); Desjardins ct al., NEJM. Epub Jun. 26, 2018; and Longo and Baden, NEJM, Epub Jun. 26, 2018).

According to the American Cancer Society (ACS), ovarian cancer ranks fifth in cancer deaths among women, accounting for more deaths than any other cancer of the female reproductive system. A woman's lifetime risk of acquiring ovarian cancer is about 1 in 78, and the lifetime chance of dying from ovarian cancer is about 1 in 108. For 2018, the ACS estimates over 22,000 ovarian cancer diagnoses and over 14,000 ovarian cancer-related deaths. The vast majority of ovarian cancers are detected at a very late stage and present with mild symptoms (e.g., hip pain). Despite improvements in therapeutic options for ovarian cancer, such as surgical cytoreduction and cytotoxic chemotherapy, there remains a need for more effective therapies, particularly for advanced disease. Oncolytic adenovirus has shown some promise targeting ovarian cancer in Phase I clinical studies (see, e.g., Kimball et al., Clin. Cancer Res., 16 (21): 5277-87 (2010); Cerullo et al., Cancer Res., 70 (11): 4297-309 (2010); and Koski et al., Mol. Ther., 18 (10): 1874-84 (2010)); however, the clinical response of these treatments remains unclear, and host secondary immune responses may inhibit their efficacy.

There remains a need for oncolytic virotherapy compositions and methods that exhibit improved tumor selectivity, antitumor activity, and tumor-specific host immune responses. The present disclosure provides such compositions and methods.

### SUMMARY

The disclosure provides a serotype 5 adenovirus or adenoviral vector comprising: (a) a nucleic acid sequence encoding a mutant E1A protein operatively linked to a first promoter that is responsive to hypoxia and inflammation, (b) a deletion of all or part of the E1B-19K region of the adenoviral genome, (c) an expression cassette located between the left inverted terminal repeat (ITR) and the E1 region of the adenovirus genome, (d) one or more exogenous nucleic acid sequences, each of which encodes an immune modulator and is operatively linked to a second promoter that is active in tumor cells, (e) one or more non-native nucleic acid sequences, each of which encodes an immune modulator and is operatively linked to the adenovirus native E3 promoter, and (f) a fiber protein comprising a serotype 3 adenovirus fiber knob domain.

The disclosure also provides a serotype 5 adenovirus or adenoviral vector comprising: (a) a nucleic acid sequence encoding a mutant E1A protein operatively linked to a secreted protein acidic and rich in cysteine (SPARC) promoter which comprises one or more hypoxia-responsive elements (HREs) and one or more nuclear factor kappa B (NF-κB) inflammation responsive elements (κBRE), (b) a deletion of all or part of the E1B-19K region of the adenoviral genome, (c) a first non-native nucleic acid sequence encoding CD40 ligand (CD40L) and a second non-native nucleic acid sequence encoding 4-1BB ligand (4-1BBL), wherein the first and second non-native nucleic acid sequences are (i) separated by an internal ribosome entry site (IRES) and (ii) operatively linked to a composite human telomerase reverse transcriptase (hTERT) promoter operatively linked to a minimal human CMV promoter and (iii) transcribed from an expression cassette located between the left ITR and the E1 region of the adenovirus genome, (d) a second exogenous nucleic acid sequence encoding IL-21 operatively linked to the adenovirus native E3 promoter, and (e) a fiber protein comprising a serotype 3 adenovirus fiber knob domain.

The disclosure also provides a composition comprising any of the aforementioned adenoviruses or adenoviral vectors, as well as a method of inducing cytotoxicity in tumor or cancer cells using the composition.

### BRIEF DESCRIPTION OF THE FIGURES

FIGS. 1A-1D are schematic diagrams illustrating several different adenovirus embodiments encompassed by the present disclosure. FIG. 1A shows the vector AdUL001.

FIG. 1B shows the vector AdUL009. FIG. 1C shows the vector AdUL016. FIG. 1D shows the vector AdUL017.

FIG. 2 is a western blot showing E1B-55K expression following infection with various adenovirus vectors encompassed by the present disclosure before and after splice correction.

FIG. 3 is a graph showing replication of AdUL001, AdUL009, AdUL016 and AdUL017 in A549 cells.

FIG. 4 is an image of a gel showing genomic stability of the SPARC promoter over 5 passages. Following each passage DNA was isolated and amplified by PCR. PCR product was loaded and evaluated by gel electrophoresis using a 0.8% standard agarose gel and 2 μL PCR reaction/lane.

FIG. 5A is a western blot showing expression of pIX antibody in various vectors described herein. The 30 kDa band (or 26 kDa on a 15% gel) is not an E1B/pIX chimera. FIG. 5B is a graph showing heat stability of the tested vectors. The graph shows the relative infectivity as a function of duration of incubation (in minutes).

FIG. 6A is a western blot showing CD40L expression and FIG. 6B is a western blot showing beta-actin expression following infection of A549 cells with various vectors described herein.

FIG. 7A is a western blot showing 4-1BBL expression (short exposure) and FIG. 7B is a western blot showing 4-1BBL expression (long exposure) following infection of RT4 cells with the various vectors described herein.

FIG. 8 is a graph showing in vitro lytic activity of various vectors described herein.

FIG. 9 is a graph showing in vivo tumor killing activity of AdUL016.

FIGS. 10A-10E are schematic diagrams illustrating several different adenovirus embodiments encompassed by the present disclosure. FIG. 10A shows the vector AdUL016.

FIG. 10B shows the vector AdUL022. FIG. 10C shows the vector AdUL025. FIG. 10D shows the vector AdUL026. FIG. 10E shows the adenovirus WT E3 region.

FIG. 11A and FIG. 11B show western blots demonstrating IL-21 expression following infection of A549 cells with various vectors described herein. FIG. 11B, bottom plot, is a western blot showing results using the beta-actin control.

FIG. 12 is a graph of ELISA results demonstrating IL-21 expression following infection of A548 cells with various vectors described herein.

FIG. 13 is a graph showing replication ability of various vectors described herein.

### DETAILED DESCRIPTION

The present disclosure is predicated, at least in part, on the development of a conditionally replicative adenovirus (CRAd) that selectively replicates in tumor-associated stromal cells and expresses immune modulator proteins. The CRAd may be used in oncolytic immunotherapy applications, particularly as an adjunct to chemotherapy and/or checkpoint inhibitor therapy.

Adenovirus is a medium-sized (90-100 nm), nonenveloped icosahedral virus containing approximately 36 kilobases (kb) of double-stranded DNA. The adenovirus capsid mediates the key interactions of the early stages of the infection of a cell by the virus and is required for packaging adenovirus genomes at the end of the adenovirus life cycle. The capsid comprises 252 capsomeres, which includes 240 hexon trimers, 12 penton base pentamer proteins, and 12 trimer fibers (Ginsberg et al., Virology, 28:782-83 (1966)). The hexon comprises three identical proteins, namely polypeptide II (Roberts et al., Science, 232:1148-51 (1986)). The penton base comprises five identical proteins and the fiber comprises three identical proteins. Proteins IIIa, VI, and IX are present in the adenoviral coat and are believed to stabilize the viral capsid (Stewart et al., Cell, 67:145-54 (1991), and Stewart et al., EMBO J., 12 (7): 2589-99 (1993)). The expression of the capsid proteins, with the exception of pIX, is dependent on the adenovirus polymerase protein. Therefore, major components of an adenovirus particle are expressed from the genome only when the polymerase protein gene is present and expressed.

Several features of adenoviruses make them ideal vehicles for transferring genetic material to cells for therapeutic applications (e.g., gene therapy, immunotherapy, or as vaccines). For example, adenoviruses can be produced in high titers (e.g., about 10¹³ particle units (pu)), and can transfer genetic material to nonreplicating and replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3:147-154 (1992)). Additionally, adenoviruses generally do not integrate into the host cell chromosome, but rather are maintained as a linear episome, thereby minimizing the likelihood that a recombinant adenovirus will interfere with normal cell function.

Over 50 serotypes of adenovirus have been identified, which are classified as subgroup A (e.g., serotypes 12, 18, and 31), subgroup B (e.g., serotypes 3, 7, 11, 14, 16, 21, 34, 35, 50, and 55), subgroup C (e.g., serotypes 1, 2, 5, and 6), subgroup D (e.g., serotypes 8, 9, 10, 13, 15, 17, 19, 20, 22-30, 32, 33, 36-39, and 42-49, 51, 53, 54, 56), subgroup E (e.g., serotype 4), subgroup F (e.g., serotypes 40 and 41), subgroup G (e.g., serotype 52). Various serotypes of adenovirus are available from the American Type Culture Collection (ATCC, Manassas, Va.).

In one embodiment, the adenovirus or adenoviral vector is a serotype 5 adenovirus or adenoviral vector (“Ad5”). The term “adenovirus,” as used herein, refers to an adenovirus that retains the ability to participate in the adenovirus life cycle and has not been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells. (2) transcription of the adenoviral genome and translation of adenovirus proteins, (3) replication of the adenoviral genome, and (4) viral particle assembly (see, e.g., Fields Virology, 5th ed., Knipe et al. (eds.). Lippincott Williams & Wilkins, Philadelphia, Pa. (2006)). The term “adenoviral vector.” as used herein, refers to an adenovirus in which the adenoviral genome has been manipulated to accommodate a nucleic acid sequence that is non-native with respect to the adenoviral genome. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

In some embodiments, the adenovirus or adenoviral vector is chimeric. A “chimeric” adenovirus or adenoviral vector may comprise an adenoviral genome that is derived from two or more (e.g., 2, 3, 4, etc.) different adenovirus serotypes. In some embodiments, a chimeric adenovirus or adenoviral vector can comprise approximately equal amounts of the genome of each of the two or more different adenovirus serotypes. When the chimeric adenovirus or adenoviral vector genome is comprised of the genomes of two different adenovirus serotypes, the chimeric adenoviral vector genome preferably comprises no more than about 95% (e.g., no more than about 90%, about 85%, about 80%, about 75%, about 70%, about 65%, about 60%, about 55%, about 50%, about 45%, or about 40%) of the genome of one of the adenovirus serotypes, with the remainder of the chimeric adenovirus genome being obtained or derived from the genome of the other adenovirus serotype. In one embodiment, the majority (i.e., greater than 50%) of the genome of the adenovirus or adenoviral vector is obtained or derived from a serotype 5 adenovirus.

As discussed above, the adenovirus and adenoviral vector disclosed herein is conditionally replicating. A conditionally-replicating adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific promoter. In such embodiments, replication requires the presence or absence of specific factors that activate or repress the promoter. Conditionally-replicating adenoviral vectors are further described in, e.g., U.S. Pat. Nos. 5,998,205; 6,824,771. Gene products essential for adenovirus replication are encoded by the E1, E2, and E4 regions of the adenoviral genome. The E1 region comprises the E1A and E1B subregions, while the E2 region comprises the E2A and E2B subregions. The E4 region comprises multiple open reading frames (ORFs), of which ORF6, and in some cases ORF3, are essential for adenovirus replication. The E3 region of the adenoviral genome does not include any replication-essential gene functions.

The early region 1A and 1B (E1A and E1B) genes encode proteins required for a productive adenovirus lytic cycle (Fields, supra). E1A is the first viral gene transcribed after infection and produces two related proteins, 243R and 289R, which induce transcription of the other early viral gene regions and stimulate infected cells to enter S-phase of the cell cycle. The E1B region encodes two major proteins, E1B19K and E1B55K. The E1B55K protein binds the cellular tumor suppressor p53 and can block p53-mediated apoptosis and inhibition of viral and cellular replication. The E1B 19K protein is a Bcl-2 homologue that interacts with Bax and inhibits apoptosis, allowing the virus to replicate longer (Sundararajan, R. and White, E, J. Virology, 75:7506-7516 (2001)). It has recently been demonstrated that the E1B proteins may not be essential for replication of oncolytic adenoviruses (Lopez et al., Mol. Ther., 20:2222-2233 (2012); and Viale et al., J. Invest. Dermatol., 133 (11): 2576-2584 (2013)). The E1A proteins have been shown to induce S-phase in infected cells by associating with p300/CBP or the retinoblastoma (Rb) protein (Howe et al., Proc. Natl. Acad. Sci. USA, 87:5883-5887 (1990); Wang et al., Mol. Cell. Biol., 11:4253-4265 (1991); Howe, J. A. and Bayley, S. T. Virology, 186:15-24 (1992)). Rb and p300 regulate the activity of E2F transcription factors, which coordinate the expression of cellular genes required for cell cycle progression (Helin, K., Curr. Opin. Genet. Dev., 8:28-35 (1998)). Thus, E1A gene products play a role in viral genome replication by driving entry of quiescent cells into the cell cycle, in part, by displacing E2F transcription factors from the retinoblastoma protein (pRb) tumor suppressor (Liu, X. and Marmorstein, R., Genes & Dev., 21:2711-2716 (2007)). Adenoviruses that conditionally replicate in certain tumor cell types typically are generated using one or a combination of the following approaches: (1) the use of tissue-specific promoters to drive expression of E1A, thereby restricting E1A-driven viral replication to specific tissues or tumors (Rodriguez et al., Cancer Res., 57:2559-2563 (1997); Alemany et al., Cancer Gene Ther. 6:21-25 (1999); Hallenbeck et al., Hum. Gene Ther., 10:1721-1733 (1999); and Howe et al., Mol. Ther. 2(5): 485-495 (2000)), (2) making mutations within the E1 region that abrogate viral protein interactions with either p53 or Rb to target tumor cells defective for those gene products, and/or (3) modification of native adenovirus tropism (Jounaidi et al., Curr. Cancer Drug Targets, 7(3): 285-301 (2007)). In one embodiment, the adenovirus or adenoviral vector described herein comprises a nucleic acid sequence encoding a mutant E1A protein. The adenovirus or adenoviral vector may comprise a nucleic acid sequence encoding any suitable mutant F1A protein, but desirably the mutant E1A protein exhibits impaired or abrogated binding to the retinoblastoma protein. The nucleic acid sequence encoding a mutant E1A protein comprises a deletion, insertion, or substitution of one or more nucleotides which renders the E1A protein encoded thereby defective for Rb binding. Mutant E1A proteins which do not bind Rb. or bind to Rb with reduced affinity, are known in the art (see, e.g., U.S. Pat. Nos. 5,801,029; 5,856,181; and 5,972,706) and may be used in connection with the disclosed adenovirus or adenoviral vector. In some embodiments, the nucleic acid sequence encoding a mutant E1A protein comprises a nucleotide sequence having one or more mutations compared to the sequence of ATGAGACATATTATCTGCCACGGAGGTGTTATTACCGAAGAAATGGCCGCCAGTCTT TTGGACCAGCTGATCGAAGAGGTACTGGCTGATAATCTTCCACCTCCTAGCCATTTT GAACCACCTACCCTTCACGAACTGTATGATTTAGACGTGACGGCCCCCGAAGATCCC AACGAGGAGGCGGTTTCGCAGATTTTTCCCGACTCTGTAATGTTGGCGGTGCAGGAA GGGATTGACTTACTCACTTTTCCGCCGGCGCCCGGTTCTCCGGAGCCGCCTCACCTTT CCCGGCAGCCCGAGCAGCCGGAGCAGAGAGCCTTGGGTCCGGTTTCTATGCCAAAC CTTGTACCGGAGGTGATCGATCTTACCTGCCACGAGGCTGGCTTTCCACCCAGTGAC GACGAGGATGAAGAGGGTGAGGAGTTTGTGTTAGATTATGTGGAGCACCCCGGGCA CGGTTGCAGGTCTTGTCATTATCACCGGAGGAATACGGGGGACCCAGATATTATGTG TTCGCTTTGCTATATGAGGACCTGTGGCATGTTTGTCTACAGTAAGTGAAAATTATG GGCAGTGGGTGATAGAGTGGTGGGTTTGGTGTGGTAATTTTTTTTTTAATTTTTACAG TTTTGTGGTTTAAAGAATTTTGTATTGTGATTTTTTTAAAAGGTCCTGTGTCTGAACC TGAGCCTGAGCCCGAGCCAGAACCGGAGCCTGCAAGACCTACCCGCCGTCCTAAAA TGGCGCCTGCTATCCTGAGACGCCCGACATCACCTGTGTCTAGAGAATGCAATAGTA GTACGGATAGCTGTGACTCCGGTCCTTCTAACACACCTCCTGAGATACACCCGGTGG TCCCGCTGTGCCCCATTAAACCAGTTGCCGTGAGAGTTGGTGGGCGTCGCCAGGCTG TGGAATGTATCGAGGACTTGCTTAACGAGCCTGGGCAACCTTTGGACTTGAGCTGTA AACGCCCCAGGCCATAAGGTGTAA (SEQ ID NO: 1). In one embodiment, the nucleic acid sequence encoding a mutant E1A protein comprises a deletion of one or more nucleotides (e.g., 1, 2, 3, 4, 5, 6, 7, 8, 9, 10 or more nucleotides) in the Rb protein binding domain of E1A. For example, in some embodiments the nucleic acid sequence encoding a mutant E1A protein comprises a deletion of one or more nucleotides (e.g., 1, 2, 3, 4, 5, 6, 7, 8, 9, 10 or more nucleotides) relative to SEQ ID NO: 1. In certain embodiments, the nucleic acid sequence encoding a mutant E1A protein comprises a deletion of 10-20 nucleotides (i.e., 10, 11, 12, 13, 14, 15, 16, 17, 18, 19, or 20 nucleotides). For example, the nucleic acid sequence encoding a mutant E1A protein may comprise the nucleic acid sequence of SEQ ID NO: 2, which includes a deletion of 15 nucleotides in the Rb protein binding domain of E1A, resulting in the deletion of amino acids 123 to 127 (TCHEA) of the Rb protein binding domain of E1A. SEQ ID NO: 2 is set forth as follows:

(SEQ ID NO: 2)
ATGAGACATATTATCTGCCACGGAGGTGTTATTACCGAAGAAATGG
CCGCCAGTCTTTTGGACCAGCTGATCGAAGAGGTACTGGCTGATA
ATCTTCCACCTCCTAGCCATTTTGAACCACCTACCCTTCACGAAC
TGTATGATTTAGACGTGACGGCCCCCGAAGATCCCAACGAGGAGG
CGGTTTCGCAGATTTTTCCCGACTCTGTAATGTTGGCGGTGCAGG
AAGGGATTGACTTACTCACTTTTCCGCCGGCGCCCGGTTCTCCGG
AGCCGCCTCACCTTTCCCGGCAGCCCGAGCAGCCGGAGCAGAGAG
CCTTGGGTCCGGTTTCTATGCCAAACCTTGTACCGGAGGTGATCG
ATCTTGGCTTTCCACCCAGTGACGACGAGGATGAAGAGGGTGAGG
AGTTTGTGTTAGATTATGTGGAGCACCCCGGGCACGGTTGCAGGT
CTTGTCATTATCACCGGAGGAATACGGGGGACCCAGATATTATGT
GTTCGCTTTGCTATATGAGGACCTGTGGCATGTTTGTCTACAGTA
AGTGAAAATTATGGGCAGTGGGTGATAGAGTGGTGGGTTTGGTGT
GGTAATTTTTTTTTTAATTTTTACAGTTTTGTGGTTTAAAGAATT
TTGTATTGTGATTTTTTTAAAAGGTCCTGTGTCTGAACCTGAGCC
TGAGCCCGAGCCAGAACCGGAGCCTGCAAGACCTACCCGCCGTCC
TAAAATGGCGCCTGCTATCCTGAGACGCCCGACATCACCTGTGTC
TAGAGAATGCAATAGTAGTACGGATAGCTGTGACTCCGGTCCTTC
TAACACACCTCCTGAGATACACCCGGTGGTCCCGCTGTGCCCCAT
TAAACCAGTTGCCGTGAGAGTTGGTGGGCGTCGCCAGGCTGTGGA
ATGTATCGAGGACTTGCTTAACGAGCCTGGGCAACCTTTGGACTT
GAGCTGTAAACGCCCCAGGCCATAAGGTGTAA.

To effect preferential replication of the adenovirus in tumor or tumor-associated cells, the nucleic acid sequence encoding the mutant E1A protein may be operatively linked to a promoter that is active in tumor cells, but not in normal cells. Such promoters are referred to herein as “tumor-specific.” “tissue-specific,” “cell-specific,” or “cancer-specific” promoters. As used herein, the term “promoter” refers to a DNA sequence that directs the binding of RNA polymerase, thereby promoting RNA synthesis. A nucleic acid sequence is “operably linked” or “operatively linked” to a promoter when the promoter is capable of directing transcription of that nucleic acid sequence. A promoter can be native or non-native to the nucleic acid sequence to which it is operably or operatively linked. Techniques for operably linking sequences together are well known in the art. A tumor-specific or cancer-specific promoter can be chosen based upon the target tissue or tumor cell type in which the nucleic acid sequence is to be expressed. A wide variety of tumor-specific promoters have been employed in conditionally replication adenovirus constructs, any of which may be used in the context of the present disclosure. Such promoters include, for example, the α-fetoprotein promoter (Hallenbeck et al., Hum Gene Ther., 10 (10):1721-33 (1999)), the prostate-specific antigen (PSA) promoter (Rodriguez et al., Cancer Res., 57(13): 2559-63 (1997)), the MUC1/DF3 promoter (Kurihara et al., J Clin Invest., 106:763-771 (2000)), the human telomerase reverse transcriptase (hTERT) promoter (Zhang et al., J Biol Chem., 287 (39): 32494-32511 (2012)), and the E2F promoter (Tsukuda et al., Cancer Res., 62:3438-3447 (2002)).

In some embodiments, the promoter may be preferentially active in tumor or cancer cells themselves; however, in other embodiments tumor-specific or cancer-specific promoters include promoters that are preferentially active in cells that are associated with a tumor or cancer (referred to herein as “tumor-associated cells” or “cancer-associated cells”). Indeed, it will be appreciated that the tumor microenvironment is a heterogeneous population of cells consisting of the tumor bulk plus supporting (or “stroma”) cells which are recruited by tumor cells from nearby endogenous host stroma. Tumor-associated stromal cells (TASCs) include, but are not limited to, vascular endothelial cells, pericytes, adipocytes, fibroblasts, and bone-marrow mesenchymal stromal cells. In certain embodiments, the nucleic acid sequence encoding the mutant E1A protein is operatively linked to a first promoter that is responsive to hypoxia and inflammation, both of which are common features of tumor cells and tumor-associated stromal cells (see. e.g., Laitala, A. and J. T. Erler. Front. Oncol., 8:189 (2018); Petrova et al., Oncogenesis. 7:10 (2018); Casazza et al., Oncogene, 33(14): 1743-1754 (2014)). Indeed. TASCs are known to secrete many pro-inflammation factors, such as, IL-6, IL-8, stromal-derived factor-1 alpha, VEGF, tenascin-C and matrix metalloproteinases (Filer et al., Discov. Med., 7(37):20-26 (2007); and Bussard et al., Breast Cancer Res., 18:84 (2016)). A promoter that is responsive to hypoxia and inflammation may be engineered by incorporating one or more hypoxia response elements (HREs) and one or more inflammation response elements. In this regard, the promoter may comprise one or more (e.g., 2, 3, 4, 5, 6, 7, 8, 9, 10, or more) HREs that are responsive to hypoxia via binding the hypoxia-inducible factor-1 (HIF-1), which is a transcription factor that plays a critical role in the cell response to oxygen deficiency, and one or more (e.g., 2, 3, 4, 5, 6, 7, 8, 9, 10, or more) NF-kB inflammation response elements (kBREs).

In one embodiment, the first promoter is a secreted protein acidic and rich in cysteine (SPARC) promoter. SPARC, also known as osteonectin or BM-40, is a multifaceted secreted glycoprotein which is expressed in many types of cells and is associated with tissue remodeling, wound repair, morphogenesis, cellular differentiation, cell migration, and angiogenesis. SPARC is differentially expressed in tumors and its surrounding stroma in various cancers. Higher levels of SPARC expression have been reported in breast cancer, melanomas, and glioblastomas. Lower levels of SPARC expression have been found in other types of cancers, such as ovarian, colorectal, pancreatic cancer, and acute myelogenous leukemia, primarily due to promoter methylation (Chen et al., Scientific Reports, 4:7035 (2014); and Tai I. T. & Tang M. J., Drug Resist Updat., 11:231-46 (2008)). In the context of the present disclosure, the SPARC promoter is engineered to include one or more hypoxia-responsive elements (HREs) and one or more nuclear factor kappa B (NF-κB) inflammation responsive elements (κBRE), so as to direct expression of the nucleic acid sequence operatively linked thereto in tumor associated stromal cells, as discussed above. A SPARC promoter suitable for use in the disclosed adenovirus or adenoviral vector is described in, for example, U.S. Pat. No. 8,436,160.

In other embodiments, it may be desirable for the adenovirus or adenoviral vector to preferentially replicate in vascular endothelial cells (ECs), particularly tumor-associated vascular endothelial cells. Vascular endothelial cells (ECs) may be ideal targets for oncolytic virus immunotherapy as they provide widespread tissue access and are the first contact surfaces following intravenous vector administration. Thus, the nucleic acid sequence encoding the mutant E1A protein may be operatively linked to a promoter that is primarily or exclusively active in endothelial cells (i.e., an “endothelial cell-specific promoter”). Any suitable endothelial cell-specific promoter known are the art may be used, including but not limited to, an ICAM-2 promoter, an endoglin promoter, a Flt-1 promoter, a roundabout 4 (ROBO4) promoter, or a Tie1 promoter. In some embodiments, the nucleic acid sequence encoding the mutant E1A protein is operatively linked to a ROBO4 promoter (described in U.S. Patent Application Publications 2016/0145643 and 2017/0159072; Okada et al., Circ Res., 100:1712-1722 (2007); and Lu et al., PLoS ONE. 8(12): e83933 (2013)), or a Tie1 promoter (described in, e.g., Korhonen, Blood, 86(5): 1828-1835 (1995)).

In some embodiments, the adenovirus or adenoviral vector may comprise a deletion, in whole or in part, of one or more regions of the adenoviral genome. In some embodiments, the adenovirus or adenoviral vector comprises a deletion of sufficient genetic material of the adenoviral genome to obliterate or impair the function of the gene (e.g., such that the function of the gene product is reduced by at least about 2-fold. 5-fold, 10-fold. 20-fold, 30-fold, or 50-fold) whose nucleic acid sequence was disrupted (e.g., deleted) in whole or in part. For the purpose of providing sufficient space in the adenoviral genome for one or more non-native nucleic acid sequences (or “transgenes”), removal of a majority of one or more gene regions may be desirable. In this regard, the adenovirus or adenoviral vector may comprise a deletion of all or part of one or more adenoviral early regions (e.g., E1, E2, and/or E3 regions), the late regions (e.g., the L1, L2, L3. L4, and L5 regions), genes involved in viral packaging (e.g., the IVa2 gene), and/or virus-associated RNAs (e.g., VA-RNA-1 and/or VA-RNA-2). In one embodiment, the adenovirus or adenoviral vector comprises a deletion of all or part of the E1B-19K region of the adenoviral genome. In some embodiments, the adenovirus or adenoviral vector comprises a deletion in all or part of the E3 region of the adenoviral genome. In some embodiments, the adenovirus or adenoviral vector comprises a deletion in all or part of the E1B-19K region and a deletion of all or part of the E3 region of the adenoviral genome. The size of the deletion may be tailored so as to retain an adenovirus or adenoviral vector whose genome closely matches the optimum genome packaging size. A larger deletion will accommodate the insertion of larger non-native nucleic acid sequences in the adenovirus or adenoviral genome. In some embodiments, the size of the deletion in the E3 region is about 2.4 Kb to about 3.0 Kb.

By removing all or part of certain regions of the adenoviral genome, for example, the E1B-19K and/or E3 regions of the adenoviral genome, the resulting adenovirus or adenoviral vector is able to accept inserts of exogenous non-native nucleic acid sequences while retaining the ability to be packaged into adenoviral capsids. Also the inactivation of E1B-19K protein by deletion or mutation increases the viral oncolytic effect of the adenovirus vector (Harrison D. et al., HumGene Ther 12:1323-1332 (2001)), as the cells are not protected against apoptosis. Thus, in another embodiment, the adenovirus or adenoviral vector comprises one or more non-native nucleic acid sequences. A non-native nucleic acid sequence can be inserted at any position in the adenoviral genome so long as insertion allows for the formation of adenovirus or the adenoviral vector particle. A “non-native” nucleic acid sequence is any nucleic acid sequence (e.g., DNA, RNA, or cDNA sequence) that is not a naturally occurring nucleic acid sequence of an adenovirus in a naturally occurring position. Thus, a non-native nucleic acid sequence can be naturally found in an adenovirus but located at a non-native position within the adenoviral genome and/or operably linked to a non-native promoter. The terms “non-native nucleic acid sequence.” “heterologous nucleic acid sequence.” and “exogenous nucleic acid sequence” are synonymous and can be used interchangeably in the context of the present disclosure. The non-native nucleic acid sequence preferably is DNA and preferably encodes a protein (e.g., one or more nucleic acid sequences encoding one or more proteins). The term “transgene” is defined herein as a non-native nucleic acid sequence that is operably linked to appropriate regulatory elements (e.g., a promoter), such that the non-native nucleic acid sequence can be expressed to produce an RNA or protein (e.g., a regulatory RNA sequence, peptide, or polypeptide). The regulatory elements (e.g., promoter) can be native or non-native to the adenovirus or adenoviral vector.

Each of the one or more non-native nucleic acids sequences desirably encodes an immune modulator. The terms “immune modulator,” “immune modulator protein,” and “immunomodulator,” are used interchangeably herein and refer to a substance or protein that affects normal immune function of an organism. In some embodiments, an immune modulator stimulates immune functions of an organism, such as by activating, boosting, or restoring immune responses. In other embodiments, an immune modulator may exert a negative effect on immune function, such as by attenuating an existing immune response or preventing the stimulation of an immune response. Immune modulators may be naturally occurring substances (e.g., proteins) or may be synthetically generated compounds. Examples of naturally occurring immune modulators include, but are not limited to, cytokines, chemokines, and interleukins. Cytokines are small proteins (˜25 kDa) that are released by a variety of cell types, typically in response to an activating stimulus, and induce responses through binding to specific receptors. Examples of cytokines include, but are not limited to, interferons (i.e., IFN-α, IFN-β, IFN-γ), leukemia inhibitory factor (LIF), oncostatin M (OSM), granulocyte-macrophage colony stimulating factor (GM-CSF), granulocyte colony stimulating factor (G-CSF), tumor necrosis factors (e.g., TNF-α), transforming growth factor (TGF)-β family members (e.g., TGF-β1 and TGF-β2). Chemokines are a class of cytokines that have chemoattractant properties, inducing cells with the appropriate receptors to migrate toward the source of the chemokine. Chemokines fall mainly into two groups: CC chemokines comprising two adjacent cysteines near the amino terminus, or CXC chemokines, in which two equivalent cysteine residues are separated by another amino acid. CC chemokines include, but are not limited to, chemokine ligands (CCL) 1 to 28, and CXC chemokines include, but are not limited to, CXC ligands (CXCL) 1 to 17. Interleukins are a structurally diverse group of cytokines which are secreted by macrophages in response to pathogens and include, for example, interleukin-1 (IL-1), IL-2, IL-6, IL-12, IL-8, and IL-21. Other cytokines, chemokines, and interleukins are known in the art and described in e.g., Cameron M. J., and Kelvin D. J., Cytokines, Chemokines and Their Receptors. In: Madame Curic Bioscience Database, Austin (TX): Landes Bioscience; 2000-2013. Available from: www.ncbi.nlm.nih.gov/books/NBK6294/. In other embodiments, an immune modulator may be synthetically or recombinantly generated. For example, an immune modulator may be a fusion protein, a chimeric protein, or any modified version of a naturally-occurring immune modulator. Recently, a recombinant fusion protein comprised of an NKG2D ligand known as orthopoxvirus major histocompatibility complex class I like protein (OMCP) and a mutated form of IL-2 with poor affinity for IL-2Rα has been developed and may be employed in the adenovirus or adenoviral vector. This fusion protein (referred to as “OMCP-mutIL-2”) potently and selectively activates IL-2 signaling only on NKG2D-bearing cells, such as natural killer (NK) cells, without broadly activating IL-2Rα-bearing cells (Ghasemi et al., Nature Communications, 7. Article No: 12878 (2016)).

In one embodiment, the adenovirus or adenoviral vector comprises a non-native nucleic acid sequence encoding the cytokine CD40 ligand (CD40L) and a non-native nucleic acid sequence encoding the cytokine 4-1BB ligand (4-1BBL). CD40L, also known as CD154, is a member of the TNF protein superfamily that is primarily expressed on activated T-cells. CD40L-CD40 interaction is crucial for the in vivo priming of Th1 T cells via the stimulation of IL-12 secretion by APC (Grewal, I. S. and Flavell, R. A., Annual Review of Immunology, 16:111-135 (1998)). In some embodiments, the nucleic acid sequence encoding CD40L may be mutated. For example, the non-native nucleic acid sequence may encode a CD40L that is resistant to metalloproteinase cleavage such that CD40L expression is retained at the cell membrane (as described in, e.g., Elmetwali et al., Molecular Cancer, 9:52 (2010)). 4-1BB ligand, also known as CD137 ligand and TNFSF9, is a transmembrane cytokine that is part of the tumor necrosis factor (TNF) ligand family. 4-1BBL is a bidirectional signal transducer that acts as a ligand for TNFRSF9, which is a costimulatory receptor molecule in T lymphocytes. 4-1BBL is expressed by activated B cells, macrophages, dendritic cells, activated T cells, neurons, and astrocytes, and its interaction with TNFRSF9 plays a role in antigen presentation development and in the generation of cytotoxic T cells. 4-1BBR is absent from resting T lymphocytes but is rapidly expressed upon antigenic stimulation. 4-1BBL is expressed in carcinoma cell lines and is thought to be involved in T cell-tumor cell interaction. The non-native nucleic acid sequence encoding 4-1BBL may also be mutated. Nucleic acid sequences encoding CD40L and 4-1BBL are publicly available and may be used in the disclosed adenovirus or adenoviral vector (see. e.g., Seyama et al., Hum Genet., 97(2): 180-5 (1996); Alderson et al., Europ. J. Immun., 24:2219-2227, 1994; NCBI Reference Sequence NG_007280.1; and NCBI Reference Sequence NM_003811.4). In some embodiments, the nucleic acid sequence encoding CD40L has at least 80% identity (e.g. at least 80%, at least 85%, at least 90%, at least 91%, at least 92%, at least 93%, at least 94%, at least 95%, at least 96%, at least 97%, at least 98%, at least 99%, or 100% sequence identity) with the sequence provided in NCBI Reference Sequence NG_007280.1. In some embodiments, the nucleic acid sequence encoding 4-1BBL has at least 80% sequence identity (e.g. at least 80%, at least 85%, at least 90%, at least 91%, at least 92%, at least 93%, at least 94%, at least 95%, at least 96%, at least 97%, at least 98%, at least 99%, or 100% sequence identity) with the sequence provided in NCBI Reference Sequence NM_003811.4. In some embodiments, the nucleic acid sequence encoding CD40L and/or the nucleic acid sequence encoding 4-1BBL encodes a recombinant form of the protein.

In other embodiments, the adenovirus or adenoviral vector may comprise a non-native nucleic acid sequence that encodes a protein that inhibits the activity of tumor associated macrophages (TAMs). Infiltration of macrophages in solid tumors is associated with poor prognosis and chemotherapy resistance in many tumors. In mouse models of cancer, macrophages promote cancer initiation and malignant progression, and at metastatic sites macrophages promote tumor cell extravasation, survival, and growth. Thus, TAMs are being investigated as potential targets for anti-cancer therapy (Cassetta, L., and J. W. Pollard, Nature Reviews Drug Discovery, 17:887-904 (2018)). In one embodiment, the non-native nucleic acid sequence may encode a protein or peptide which binds to, and inhibits signaling mediated by, the colony-stimulating factor 1 receptor (CSF1R), which is a canonical marker expressed by macrophages. For example, the non-native nucleic acid sequence may encode an antibody, or antigen-binding fragment thereof, that binds to and inhibits the activity of CSF1R. Several antibodies which specifically bind to and inhibit the activity of CSF1R are known in the art and may be encoded by the non-native nucleic acid sequence, including, for example emactuzumab (also referred to as RG-7155, see, e.g., ClinicalTrials.gov Identifier NCT02760797, “A Study of Emactuzumab and RO7009789 Administered in Combination in Participants with Advanced Solid Tumors”). Other receptors besides CSF1R that are expressed by tumor associated macrophages may be targeted by the adenovirus or adenoviral vector.

The one or more non-native nucleic acid sequences ideally are operatively linked to a second promoter that is active in tumor cells. Any suitable promoter that can direct transcription in a tumor or cancer cell may be employed. For example, the promoter may be a constitutive promoter, such as a CMV, RSV, SV40, EF2 or similar viral or mammalian promoter. More preferably the promoter is a “tumor-specific” promoter, as described herein. The one or more non-native nucleic acid sequences may be operatively linked to any tumor-specific promoter known in the art, such as those described herein. Prostate-specific antigen (PSA), cyclooxygenase-2 (Cox2), and human telomerase reverse transcriptase (TERT) promoters are examples of promoter sequences that can be used to confer selective viral replication to target tissues. In one embodiment, the one or more non-native nucleic acid sequences are operatively linked to a composite human telomerase reverse transcriptase (hTERT) promoter operatively linked to a minimal human CMV promoter (Davis J J et al., Cancer Gene Ther 13:720-3 (2006). Numerous studies have demonstrated that hTERT expression is highly specific to cancer cells and tightly associated with telomerase activity, while the other telomerase subunits are constitutively expressed both in normal and cancer cells (Takakura et al., Cancer Res; 58:1558-61 (1998); Kyo et al., Int. J Cancer, 80:60-3 (1999); Kanaya et al., Int J Cancer, 78:539-43 (1998); Kyo et al., Int. J Cancer. 80:804-9 (1999); Kyo et al., Cancer Science, 99 (8): 1528-1538 (2008); and Zhang et al., J Biol Chem., 287 (39): 32494-32511 (2012)). The 5′ promoter region of the hTERT gene has been characterized by several groups (see, e.g., Takakura et al., Cancer Res., 59:551-7 (1999); Horikawa et al., Cancer Res., 59:826-30 (1999); and Cong et al., Hum Mol Genet; 8:137-42 (1999)), and deletion analysis of the promoter identified a 260 bp core promoter region essential for cancer-specific transcriptional activation. Within the core promoter region, several distinct transcription factor-binding sights are present, including E-boxes (CACGTG (SEQ ID NO: 6)) and GC-boxes (GGGCGG (SEQ ID NO: 7)).

In embodiments where the adenovirus or adenoviral vector comprises two or more non-nucleic acid sequences, the two or more non-native nucleic acid sequences may be operatively linked to the same promoter (e.g., to form a “bicistronic,” “multicistronic,” “or polycistronic” sequence), the two or more non-native nucleic acid sequences may be operatively linked to separate identical promoters, or the two or more non-native nucleic acid sequences may be operatively linked to separate and different promoters. When the adenovirus or adenoviral vector comprises a non-native nucleic acid sequence encoding CD40L and a non-native nucleic acid sequence encoding 4-1BBL (also known as TNF superfamily member 9, or TNFSF9), both nucleic acid sequences may be operatively linked to the same tumor-specific promoter. When two or more nucleic acid sequences are operatively linked to a single promoter, the nucleic acid sequences desirably are separated by an internal ribosomal entry site (IRES) or a 2A peptide (or 2A peptide-like) sequence. IRESs allow for uncoupling of translation of each coding sequence thereby avoiding the generation of inactive proteins and incorrect subcellular targeting. Promoter interference or suppression also are alleviated through the use of IRESs (see, e.g., Vagner et al., EMBO Rep., 2:893-898 (2001)). 2A self-cleaving peptides were first identified in Picornaviruses as an oligopeptide (usually 19-22 amino acids) located between two proteins in some members of the picornavirus family. 2A peptides have since been identified in other viruses. Advantages of using 2A peptides for multicistronic gene expression include, for example, their small size and their ability for efficient coexpression of genes that are placed between them. Indeed, genes placed downstream of different 2A peptide sequences can induce higher levels of expression as compared to IRESs (see, e.g., Szymczak, A. L. & Vignali, D. A., Expert Opin Biol Ther., 5:627-638 (2005)).

In some embodiments, the adenovirus or adenoviral vector comprises one or more non-native nucleic acid sequences encoding an immune modulator operatively linked to the adenovirus native E3 promoter. In some embodiments, the adenovirus or adenoviral vector comprises one or more non-native nucleic acid sequences encoding an immune modulator operatively linked to a second promoter, as described above, and one or more non-native nucleic acid sequences encoding an immune modulator operatively linked to the adenovirus native E3 promoter. In some embodiments, the second promoter is a composite human telomerase reverse transcriptase (hTERT) promoter operatively linked to a minimal human CMV promoter. In some embodiments the adenovirus or adenoviral vector comprises one or more non-native nucleic acid sequences encoding an immune modulator operatively linked to a composite human telomerase reverse transcriptase (hTERT) promoter operatively linked to a minimal human CMV promoter, and one or more non-native nucleic acid sequences encoding an immune modulator operatively linked to the adenovirus native E3 promoter. In some embodiments, the one or more non-native nucleic acid sequences operatively linked to the second promoter (e.g., the composite human telomerase reverse transcriptase (hTERT) promoter operatively linked to a minimal human CMV promoter) comprise a first exogenous nucleic acid sequence encoding CD40L and a second exogenous nucleic acid sequence encoding 4-1BBL. In some embodiments, the first and second exogenous nucleic acid sequences are separated by an IRES.

In some embodiments, the one or more non-native nucleic acid sequences operatively linked to the adenovirus native E3 promoter comprise a third exogenous nucleic acid sequence encoding IL-21. In some embodiments, the one or more non-native nucleic acid sequences operatively linked to the adenovirus native E3 promoter comprise a third exogenous nucleic acid sequence encoding human IL-21. In some embodiments, the third exogenous nucleic acid sequence encodes a recombinant human IL-21. In some embodiments, the third exogenous nucleic acid sequence encoding human IL-21 encodes a human IL-21 having the following sequence: MRSSPGNMERIVICLMVIFLGTLVHKSSSQGQDRHMIRMRQLIDIVDQLKNYVNDLVP EFLPAPEDVETNCEWSAFSCFQKAQLKSANTGNNERIINVSIKKLKRKPPSTNAGRRQKH RLTCPSCDSYEKKPPKEFLERFKSLLQKMIHQHLSSRTHGSEDS (SEQ ID NO: 8). In some embodiments, the third exogenous nucleic acid sequence encoding human IL-21 encodes a human IL-21 having the following sequence: MRSSPGNMERIVICLMVIFLGTLVHKSSSQGQDRHMIRMRQLIDIVDQLKNYVNDL VP EFLPAPEDVETNCEWSAFSCFQKAQLKSANTGNNERIINVSIKKLKRKPPSTNAGRRQKH RLTCPSCDSYEKKPPKEFLERFKSLLQKVSTLSFI (SEQ ID NO: 9). Amino acids in bold represent a signal peptide that is cleaved during processing. In some embodiments, the third exogenous nucleic acid sequence encodes human IL-21 lacking this signal peptide. In some embodiments the third exogenous nucleic acid sequence encodes human IL-21 having the following sequence: HKSSSQGQDRHMIRMRQLIDIVDQLKNYVNDLVPEFLPAPEDVETNCEWSAFSCFQKAQ LKSANTGNNERIINVSIKKLKRKPPSTNAGRRQKHRLTCPSCDSYEKKPPKEFLERFKSLL QKMIHQHLSSRTHGSEDS (SEQ ID NO: 10). In some embodiments the third exogenous nucleic acid sequence encodes human IL-21 having the following sequence: HKSSSQGQDRHMIRMRQLIDIVDQLKNYVNDLVPEFLPAPEDVETNCEWSAFSCFQKAQ LKSANTGNNERIINVSIKKLKRKPPSTNAGRRQKHRLTCPSCDSYEKKPPKEFLERFKSLL QKVSTLSFI (SEQ ID NO: 11). In some embodiments, the third exogenous nucleic acid sequence encodes human IL-21 having the following sequence: MQDRHMIRMR QLIDIVDQLKNYVNDLVPEFLPAPEDVETNCEWSAFSCFQKAQLKSANTGNNERIINVSI KKLKRKPPSTNAGRRQKHRLTCPSCDSYEKKPPKEFLERFKSLLQKMIHQHLSSRTHGSE DS (SEQ ID NO: 12) In some embodiments, the third exogenous nucleic acid sequence encodes an IL-21 having at least 80% sequence identity (e.g. at least 80%, at least 85%, at least 90%, at least 91%, at least 92%, at least 93%, at least 94%, at least 95%, at least 96%, at least 97%, at least 98%, at least 99%, or 100% sequence identity) with SEQ ID NO: 8, SEQ ID NO: 9, SEQ ID NO: 10, SEQ ID NO: 11, or SEQ ID NO: 12.

In some embodiments, the one or more non-native nucleic acid sequences encoding an immune modulator are located in the expression cassette located between the left ITR and the E1 region of the adenovirus genome. For example, in some embodiments the one or more non-native nucleic acid sequences encoding an immune modulator operatively linked to a second promoter (e.g., the composite human telomerase reverse transcriptase (hTERT) promoter operatively linked to a minimal human CMV promoter) are located in an expression cassette located between the left ITR and the E1 region of the adenovirus genome. In some embodiments, the one or more non-native nucleic acid sequences encoding an immune modulator are located in the deleted E3 region of the adenoviral genome. For example, in some embodiments the one or more nucleic acid sequences encoding an immune modulator operatively linked to the adenovirus native E3 promoter are located in the deleted E3 region of the adenoviral genome. In some embodiments, one or more non-native nucleic acid sequences encoding an immune modulator are located in the deleted E3 region of the adenoviral genome and one or more non-native nucleic acid sequences encoding an immune modulator are located in the expression cassette located between the left ITR and the E1 region of the adenovirus genome. For example, in some embodiments the one or more nucleic acid sequences encoding an immune modulator operatively linked to the second promoter (e.g. the composite human telomerase reverse transcriptase (hTERT) promoter operatively linked to a minimal human CMV promoter) are located in the expression cassette located between the left ITR and the E1 region of the adenovirus genome and the one or more nucleic acid sequences encoding an immune modulator operatively linked to the adenovirus native E3 promoter are located in the deleted E3 region of the adenoviral genome. In some embodiments, a first exogenous nucleic acid sequence encoding CD40L and a second exogenous nucleic acid sequence encoding 4-1BBL arc operatively linked to a second promoter (e.g. the composite human telomerase reverse transcriptase (hTERT) promoter operatively linked to a minimal human CMV promoter) and are located in the expression cassette located between the left ITR and the E1 region of the adenovirus genome, and a third exogenous nucleic acid sequence encoding IL-21 under a native adenovirus E3 promoter are located in the deleted E3 region of the adenoviral genome.

In certain embodiments, the adenovirus or adenoviral vector comprises at least one modified capsid protein. The adenovirus capsid mediates the key interactions of the early stages of the infection of a cell by the virus and is required for packaging adenovirus genomes at the end of the adenovirus life cycle. The capsid comprises 252 capsomeres, which includes 240 hexons, 12 penton base proteins, and 12 fibers (Ginsberg et al., Virology, 28:782-83 (1966)). In one embodiment, one or more capsid proteins (also referred to herein as “coat” proteins) of the adenovirus or adenoviral vector can be manipulated to alter the binding specificity or recognition of the virus or vector for a receptor on a potential host cell. It is well known in the art that almost immediately after intravenous administration, adenovirus vectors are predominantly sequestered by the liver, with clearance of Ad5 from the bloodstream and accumulation in the liver occurring within minutes of administration (Alemany et al., J. Gen. Virol., 81:2605-2609 (2000)). Liver sequestration of adenovirus is primarily due to the abundance of the native coxsackie and adenovirus receptor (CAR) on hepatocytes. Thus, the manipulation of capsid proteins may broaden the range of cells infected by the adenovirus or adenoviral vector or enable targeting of the adenoviral vector to a specific cell type. For example, one or more capsid proteins may be manipulated so as to target the adenovirus or adenoviral vector protein to tumor cells or tumor-associated cells. Such manipulations can include deletions of the fiber, hexon, and/or penton proteins (in whole or in part), insertions of various native or non-native ligands into portions of the capsid proteins, and the like.

In some embodiments, the adenovirus or adenoviral vector comprises a modified fiber protein. The adenovirus fiber protein is a homotrimer of the adenoviral polypeptide IV that has three domains: the tail, shaft, and knob. (Devaux et al., J. Molec. Biol., 215:567-88 (1990), Yeh et al., Virus Res., 33:179-98 (1991)). The fiber protein mediates primary viral binding to receptors on the cell surface via the knob and the shaft domains (Henry et al., J. Virol., 68 (8): 5239-46 (1994)). The amino acid sequences for trimcrization are located in the knob, which appears necessary for the amino terminus of the fiber (the tail) to properly associate with the penton base (Novelli et al., Virology, 185:365-76 (1991)). In addition to recognizing cell receptors and binding the penton base, the fiber contributes to serotype identity. Fiber proteins from different adenoviral serotypes differ considerably (see. e.g., Green et al., EMBO J., 2:1357-65 (1983), Chroboczek et al., Virology. 186:280-85 (1992), and Signas et al., J. Virol., 53:672-78 (1985)). Thus, the fiber protein has multiple functions key to the life cycle of adenovirus.

The fiber protein is “modified” in that it comprises a non-native amino acid sequence in addition to or in place of a wild-type fiber amino acid sequence of the serotype 5 adenovirus or adenoviral vector. Serotype 5 adenovirus entry into cells is mediated by an initial binding step to its primary receptor, the coxsackie and adenovirus receptor (CAR). CAR exhibits reduced expression on the surface of many neoplastic cells, however. In contrast, most cells express high levels of the receptors for serotype 3 adenovirus, CD46 and Desmoglein-2 (DSG-2). Thus, modification of the serotype 5 adenovirus fiber protein has been shown to substantially improve infectivity for human tumor cells. In one embodiment, at least a portion of the wild-type fiber protein (e.g., the fiber tail, the fiber shaft, the fiber knob, or the entire fiber protein) of the disclosed serotype 5 adenovirus or adenoviral vector desirably is removed and replaced with a corresponding portion of a fiber protein from an adenovirus of a different serotype (such as those described herein). In one embodiment, the knob domain of the fiber protein of the disclosed serotype 5 adenovirus or adenoviral vector is removed and replaced with a corresponding fiber knob domain of a different adenovirus serotype. For example, the fiber protein of the serotype 5 adenovirus or adenoviral vector described may comprise a knob domain from a serotype 3 adenovirus. Other regions of the serotype 5 adenovirus fiber protein (i.e., the shaft and/or tail domains) may be removed and replaced with corresponding regions from other adenovirus serotypes (e.g., serotype 3). In one embodiment, the entire wild-type fiber protein of the serotype 5 adenovirus or adenoviral vector is replaced with the entire fiber protein of a serotype 3 adenovirus. Exchanging regions of serotype 5 adenovirus fiber protein for corresponding serotype 3 regions is described in, e.g., U.S. Pat. Nos. 5,846,782 and 7,297,542. Amino acid sequences of adenovirus serotype 3 fiber protein have been characterized and are publicly available (see, e.g., Signäs, et al., J Virol., 53(2): 672-678 (1985); and UniProtKB/Swiss-Prot Accession No. P04501).

In another embodiment, at least a portion of the wild-type fiber protein of the disclosed serotype 5 adenovirus or adenoviral vector is removed and replaced with a non-adenovirus (i.e., heterologous) amino acid sequence. For example, the knob domain of the fiber protein of the serotype 5 adenovirus or adenoviral vector may be removed and replaced with an amino acid sequence or motif that has been synthetically or recombinantly generated. A few heterologous peptides have been introduced into the fiber knob domain to re-target the adenovirus, including oligo lysine. FLAG, RGD-4C RGS (His) 6, and HA epitope. Due to the rather complex structure of the fiber knob domain, however, any heterologous peptide or amino acid sequence introduced into the fiber knob should not destabilize the fiber, which would render it incapable of trimerization and, hence, non-functional. Thus, any suitable heterologous amino acid sequence may be incorporated into the fiber knob domain, so long as the fiber protein is able to trimerize. In some embodiments, the fiber knob of the adenovirus or adenoviral vector described herein is removed and replaced with a trimerization motif and a receptor-binding ligand. For example, the fiber knob of the adenovirus or adenoviral vector described herein can be removed and replaced with a heterologous protein comprising the tail and two amino-terminal repeats of the shaft domain of the Ad5 fiber protein genetically fused with a truncated form of the bacteriophage T4 fibritin protein, and a ligand, as described in U.S. Pat. No. 6,815,200 and Krasnykh et al., J. Virol., 75(9): 4176-4183 (2001). Other examples of heterologous proteins that can replace the fiber knob region of the adenovirus or adenoviral vector include an isoleucine trimerization motif and the neck region peptide from human lung surfactant D.

The ligand may be any suitable molecule or peptide that specifically recognizes a cell surface protein that is not a native adenovirus receptor. Examples of suitable ligands include, but are not limited to, physiological ligands, anti-receptor antibodies, and cell-specific peptides. In one embodiment, the ligand is an antibody, antibody fragment, or a derivative of an antibody. In one embodiment, the ligand is an antibody fragment. Examples of antibody fragments include, but are not limited to. (i) a Fab fragment, which is a monovalent fragment consisting of the VL, VH, CL, and CH domains, (ii) a F(ab′)2 fragment, which is a bivalent fragment comprising two Fab fragments linked by a disulfide bridge at the hinge region. (iii) a Fv fragment consisting of the VL and VH domains of a single arm of an antibody. (iv) a Fab′ fragment, which results from breaking the disulfide bridge of an F(ab′)2 fragment using mild reducing conditions. (v) a disulfide-stabilized Fv fragment (dsFv), and (vi) a single domain antibody (sdAb), which is an antibody single variable region domain (VH or VL) polypeptide that specifically binds an antigen. In one embodiment, the ligand is a single domain antibody derived from an antibody produced by a camelid (i.e., camels and alpacas). Camelids produce nonconventional antibodies that consist of only the two heavy-chains (no light-chains) as the basis of antigen (Ag) recognition and binding, and these antibodies have been utilized in adenovirus-mediated gene therapy (Revets et al., Expert Opin. Biol. Ther., 5:111-124 (2005)). Camelid sdAbs possess characteristics ideal for retargeting of adenovirus, such as cytosolic stability allowing functional incorporation into the adenovirus capsid and compatibility with phage biopanning selection to allow target cell specificity (Beatty, M. S. and D. T. Curiel, Adv. Cancer Res., 115:39-67 (2012)). Indeed, camelid sdAb fragments have been demonstrated to be robust ligands for targeting adenovirus vectors (see. e.g., Kaliberov et al., Lab Invest., 94(8): 893-905 (2014); van Erp et al., Mol. Ther. Oncolytics, 2:15001 (2015); and U.S. Patent Application Publication 2017/0044269). It will be appreciated that the choice of camelid single domain antibody used in the modified fiber knob domain will depend on the desired cell type for adenovirus targeting. When the adenovirus or adenoviral vector is targeted to tumor or cancer cells, as described herein, the camelid sdAb ideally specifically binds to a receptor that is aberrantly expressed on the surface of tumor or cancer cells (also referred to as “tumor-specific” or “cancer-specific” receptors). Numerous tumor- or cancer-cell specific cell surface receptors are known in the art, and include, but are not limited to, HER2/neu, estrogen receptors, biotin receptor, c(RGD-K), epidermal growth factor receptor (EGFR), endothelin receptor B, fibroblast growth factor receptor (FGFR), somatostatin receptors, vasoactive intestinal peptide (VIP) receptors, cholecystokinin (CCK) receptors, bombesin/gastrin-releasing peptide (GRP) receptors, neurotensin receptors, substance P, neuropeptide Y, α-melanocyte-stimulating hormone (α-MSH), calcitonin, endothelin, carcinoembryonic antigen (CEA), and CD276 (B7-H3).

In other embodiments, the fiber protein comprises a non-native amino acid sequence that binds αvβ3, αvβ5, or αvβ6 integrins. Adenoviruses displaying ligands specific for avß3 integrin, such as an RGD motif, infect cells with a greater number of αvβ3 integrin moieties on the cell surface compared to cells that do not express the integrin to such a degree, thereby targeting the vectors to specific cells of interest. For example, the adenovirus or adenoviral vector may comprise a chimeric fiber protein comprising a non-native amino acid sequence comprising an RGD motif including, but not limited to, CRGDC (SEQ ID NO: 3), CXCRGDCXC (SEQ ID NO: 4), wherein X represents any amino acid, and CDCRGDCFC (SEQ ID NO: 5). The RGD motif can be inserted into the adenoviral fiber knob region, preferably in an exposed loop of the adenoviral knob, such as the HI loop.

Other regions of the serotype 5 adenovirus fiber protein (i.e., the shaft and/or tail domains) may be removed and replaced with corresponding regions from other adenovirus serotypes or non-adenovirus peptides. Any suitable amino acid residue(s) of the wild-type fiber protein of the disclosed serotype 5 adenovirus or adenoviral vector can be modified or removed, so long as viral capsid assembly is not impeded. Similarly, amino acids can be added to the fiber protein as long as the fiber protein retains the ability to trimerize. Such modified fiber proteins also are referred to as “chimeric” fiber proteins, as they comprise amino acid sequences obtained or derived from two different adenovirus serotypes.

In some embodiments, the adenovirus or adenoviral vector comprises a modified hexon protein. The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, the hexon protein is a primary target for modification to reduce neutralization of adenoviral vectors (see, e.g., Gall et al., J. Virol., 72:10260-264 (1998), and Rux et al., J. Virol., 77 (17): 9553-9566 (2003)). The major structural features of the hexon protein are shared by adenoviruses across serotypes, but the hexon protein differs in size and immunological properties between serotypes (Jornvall et al., J. Biol. Chem., 256 (12): 6181-6186 (1981)). A comparison of 15 adenovirus hexon proteins revealed that the predominant antigenic and serotype-specific regions of the hexon protein appear to be in loops 1 and 2 (i.e., LI or 11, and LII or 12, respectively), within which are seven to nine discrete hypervariable regions (HVR1 to HVR 7 or HVR9) varying in length and sequence between adenoviral serotypes (Crawford-Miksza et al., J. Virol., 70 (3): 1836-1844 (1996), and Bruder et al., PLoS ONE, 7 (4): e33920 (2012)).

The hexon protein is “modified” in that it comprises a non-native amino acid sequence in addition to or in place of a wild-type hexon amino acid sequence of the scrotype 5 adenovirus or adenoviral vector. The Ad5 hexon protein mediates liver sequestration of the virus (Waddington et al., Cell. 132:397-409 (2008); Vigant et al., Mol. Ther., 16:1474-1480 (2008); and Kalyuzhniy et al., Proc. Natl. Acad. Sci. USA, 105:5483-5488 (2008)), and modification of the hexon protein, specifically within the hypervariable 5 (HVR5) and 7 (HVR7) regions, has been shown to mitigate the endogenous liver sequestration of serotype 5 adenovirus particles (see, e.g., Alba et al., Blood, 114:965-971 (2009); Shashkova et al., Mol. Ther., 17:2121-2130 (2009); and Short et el., Mol Cancer Ther., 9 (9): 2536-2544 (2010)). In one embodiment, at least a portion of the wild-type hexon protein (e.g., the entire hexon protein) of the disclosed serotype 5 adenovirus or adenoviral vector desirably is removed and replaced with a corresponding portion of a hexon protein from an adenovirus of a different serotype (such as those described herein). In one embodiment, the hexon protein of the serotype 5 adenovirus or adenoviral vector comprises one or more hypervariable regions (HVRs) from an adenovirus of a different serotype. For example, the hexon protein of the serotype 5 adenovirus or adenoviral vector comprises one or more (e.g., one, two, three, four, five, six, or all seven) hypervariable regions (HVRs) from a serotype 3 or serotype 11 adenovirus. In other words, one or more of the HVRs of the hexon protein of the disclosed serotype 5 adenovirus or adenoviral vector may be removed and replaced with one or more corresponding HVRs from a serotype 3 or serotype 11 adenovirus. In one embodiment, the entire wild-type hexon protein of the serotype 5 adenovirus or adenoviral vector is replaced with the entire hexon protein of a serotype 3 or serotype 11 adenovirus.

Hexon protein amino acid sequences of multiple strains of adenovirus serotype 3 have been characterized and are publicly available (see, e.g., Haque et al., PLoS ONE, 13 (4): e0196263. doi.org/10.1371/journal.pone.0196263 (2018)). However, any suitable amino acid residue(s) of the wild-type hexon protein of the disclosed serotype 5 adenovirus or adenoviral vector can be modified or removed, so long as viral capsid assembly is not impeded. Similarly, amino acids can be added to the hexon protein as long as the structural integrity of the capsid is maintained. Such modified hexon proteins also are referred to as “chimeric” hexon proteins, as they comprise amino acid sequences obtained or derived from two different adenovirus serotypes.

Methods for generating modified (e.g., chimeric) adenovirus hexon and fiber proteins known in the art can be used in the context of the present disclosure. Such methods are described in, for example, U.S. Pat. Nos. 5,543,328; 5,559,099; 5,712,136; 5,731,190; 5,756,086; 5,770,442; 5,846,782; 5,871,727; 5,885,808; 5,922,315; 5,962,311; 5,965,541; 6,057,155; 6,127,525; 6,153,435; 6,329,190; 6,455,314; 6,465,253; 6,576,456; 6,649,407; 6,740,525; and 6,815,200.

The adenovirus or adenoviral described herein may be produced in cell lines suitable for propagation of conditionally replicating adenoviruses, including, for example, KB cells, HeLa cells, and A549 cells (see, e.g., Lawrence and Ginsberg, J. Virol., 1:851-867 (1967); Green and Pina, Virology, 20:199-207 (1963); Wold, Adenovirus Methods and Protocols (Humana Press, Totowa, NJ) (1999)). Methods for the production and purification of adenoviruses and adenoviral vectors are described in, e.g., U.S. Pat. No. 6,194,191, and International Patent Application Publications WO 99/54441, WO 98/22588, WO 98/00524, WO 96/27677, and WO 2003/078592.

While a variety of adenoviruses or adenoviral vectors comprising various combinations of promoters, non-native nucleic acid sequences, adenoviral genome deletions, and chimeric fiber proteins are encompassed by the present disclosure, particular embodiments include adenoviruses or adenoviral vectors comprising the following: (1) (a) a nucleic acid sequence encoding a mutant E1A protein operatively linked to the engineered SPARC promoter described herein that is responsive to hypoxia and inflammation, (b) a deletion of all or part of the E1B-19K region of the adenoviral genome, (c) a first exogenous nucleic acid sequence encoding CD40L and a second exogenous nucleic acid sequence encoding 4-1BBL, which are operatively linked to a composite hTERT promoter (in 5′-3′ or 3′-5′ orientation) operatively linked to a minimal human CMV promoter and transcribed from an expression cassette located between the left ITR and the E1 region of the adenovirus genome, (d) a third exogenous nucleic acid sequence encoding IL-21 operatively linked to the adenovirus native E3 promoter, and (e) a fiber protein comprising a serotype 3 adenovirus fiber knob domain.

The disclosure provides a composition comprising the adenovirus or adenoviral vector described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the adenovirus or adenoviral vector. Any suitable carrier can be used within the context of the disclosure, and such carriers are well known in the art. The choice of carrier will be determined, in part, by the particular use of the composition (e.g., administration to an animal) and the particular method used to administer the composition. In some embodiments, the pharmaceutical composition can be sterile.

Suitable compositions include aqueous and non-aqueous isotonic sterile solutions, which can contain antioxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the adenovirus or adenoviral vector is part of a composition formulated to protect the adenovirus or adenoviral vector from damage prior to administration. For example, the composition can be formulated to reduce loss of the adenovirus or adenoviral vector on devices used to prepare, store, or administer the adenovirus or adenoviral vector, such as glassware, syringes, or needles. The composition can be formulated to decrease the light sensitivity and/or temperature sensitivity of the adenovirus or adenoviral vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the adenovirus or adenoviral vector and facilitate its administration. Formulations for adenovirus or adenoviral vector-containing compositions are further described in, for example, U.S. Pat. Nos. 6,225,289, 6,514,943, and 7,456,009 and International Patent Application Publication WO 2000/034444.

In addition, one of ordinary skill in the art will appreciate that the adenovirus or adenoviral vector can be present in a composition with other therapeutic or biologically-active agents. For example, factors that control inflammation, such as ibuprofen or steroids, can be part of the composition to reduce swelling and inflammation associated with in vivo administration of the adenovirus or adenoviral vector. Antibiotics, i.e., microbicides and fungicides, can be present to treat existing infection and/or reduce the risk of future infection, such as infection associated with virus administration.

The dose of adenovirus or adenoviral vector present in the composition will depend on a number of factors, including the intended target tissue, the extent of any side-effects, the particular route of administration, and the like. The dose ideally comprises an “effective amount” of an adenovirus or adenoviral vector, i.e., a dose of adenovirus or adenoviral vector which provokes a desired response in a recipient (e.g., a human). Desirably, a single dose of adenovirus or adenoviral vector comprises at least about 1×10⁷ particles (which also is referred to as particle units (pu) or virus particles (vp)) of the adenoviral vector. The dose is at least about 1×10⁸ particles (e.g., about 1×10⁹-1×10¹⁴ particles), and preferably at least about 1×10¹⁰ particles, (e.g., about 1×10¹⁰-1×10¹² particles) of the adenovirus or adenoviral vector. Alternatively, the dose comprises no more than about 1×10¹⁴ particles, preferably no more than about 1×10¹³ particles, and more preferably no more than about 1×10¹² particles. In other words, a single dose of adenoviral vector can comprise, for example, about 1×10⁷ virus particles, 2×10⁷ vp. 4×10⁷ vp. 1×10⁸ vp. 2×10⁸ vp. 4×10⁸ vp. 1×10⁹ vp. 2×10⁹ vp. 4×10⁹ vp. 1×10¹⁰ vp. 2×10¹⁰ vp. 4×10¹⁰ vp, 1×10¹¹ vp, 2×10¹¹ vp, 4×10¹¹ vp. 1×10¹² vp. 2×10¹² vp. 4×10¹² vp, 1×10¹³ vp, 2×10¹³ vp, 4×10¹³ vp, or 1×10¹⁴ vp of the adenovirus or adenoviral vector.

The disclosure also provides method of inducing cytotoxicity in tumor cells which comprises contacting tumor cells with the above-described composition. The term “tumor,” as used herein, refers to an abnormal mass of tissue that results when cells divide more than they should or do not die when they should. In the context of the present disclosure, the term tumor may refer to tumor cells and tumor-associated stromal cells (as described above). Tumors may be benign and non-cancerous if they do not invade nearby tissue or spread to other parts of the organism. In contrast, the terms “malignant tumor,” “cancer.” and “cancer cells” may be used interchangeably herein and refer to a tumor comprising cells that divide uncontrollably and can invade nearby tissues. Cancer cells also can spread or “metastasize” to other parts of the body through the blood and lymph systems. The disclosed method ideally induces cytotoxicity in malignant tumor cells or cancer cells. The malignant tumor cells or cancer cells may be from a carcinoma (cancer arising from epithelial cells), a sarcoma (cancer arising from bone and soft tissues), a lymphoma (cancer arising from lymphocytes), a blood cancer (e.g., myeloma or leukemia), a melanoma, or brain and spinal cord tumors. The malignant tumor or cancer cells can be located in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. More particularly, cancers of the digestive system can affect the esophagus, stomach, small intestine, colon, rectum, anus, liver, gall bladder, and pancreas. Cancers of the respiratory system can affect the larynx, lung, and bronchus and include, for example, non-small cell lung carcinoma. Cancers of the reproductive system can affect the uterine cervix, uterine corpus, ovaries, vulva, vagina, prostate, testis, and penis. Cancers of the urinary system can affect the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). In one embodiment, the tumor cells are ovary cells, such as ovarian cancer cells.

An agent is “cytotoxic” and induces “cytotoxicity” if the agent (e.g., the adenovirus or adenoviral vector described herein) kills or inhibits the growth of cells, particularly cancer cells. In some embodiments, for example, cytotoxicity includes preventing cancer cell division and growth, as well as reducing the size of a tumor or cancer. Cytotoxicity of tumor cells may be measured using any suitable cell viability assay known in the art, such as, for example, assays which measure cell lysis, cell membrane leakage, and apoptosis. For example, methods including but not limited to trypan blue assays, propidium iodide assays, lactate dehydrogenase (LDH) assays, tetrazolium reduction assays, resazurin reduction assays, protease marker assays, 5-bromo-2′-deoxy-uridine (BrdU) assays, and ATP detection may be used. Cell viability assay systems that are commercially available also may be used and include, for example, CELLTITER-GLO® 2.0 (Promega, Madison, WI), VIVAFIX™ 583/603 Cell Viability Assay (Bio-Rad, Hercules, CA); and CYTOTOX-FLUOR™ Cytotoxicity Assay (Promega, Madison, WI).

Ideally, the disclosed method promotes inhibition of tumor cell proliferation, the eradication of tumor cells, and/or a reduction in the size of at least one tumor such that a mammal (e.g., a human) is treated for cancer. By “treatment of cancer” is meant alleviation of cancer in whole or in part. In one embodiment, the disclosed method reduces the size of a tumor at least about 20% (e.g., at least about 25%, 30%, 35%, 40%, 45%, 50%, 55%, 60%, 65%, 70%, 75%, 80%, 85%, 90%, or 95%). Ideally, the tumor is completely eliminated.

The tumor cells may be contacted with the adenovirus composition in vitro or in vivo. The term “in vivo” refers to a method that is conducted within living organisms in their normal, intact state, while an “in vitro” method is conducted using components of an organism that have been isolated from its usual biological context. When the cell is contacted with the composition in vitro, the cell may be any suitable prokaryotic or eukaryotic cell. When the cell is contacted with the composition in vivo, the composition may be administered to an animal, such as a mammal, particularly a human, using standard administration techniques and routes. Suitable administration routes include, but are not limited to, oral, intravenous, intraperitoneal, subcutaneous, pulmonary, transdermal, intramuscular, intranasal, buccal, sublingual, or suppository administration. The composition preferably is suitable for parenteral administration. The term “parenteral.” as used herein, includes intravenous, intramuscular, subcutaneous, rectal, vaginal, and intraperitoneal administration. In other embodiments, the composition may be administered to a mammal using systemic delivery by intravenous, intramuscular, intraperitoneal, or subcutaneous injection.

While administration of a single dose of the adenovirus or adenoviral vector can be accomplished through a single application of the composition (e.g., a single injection to the target tissue), in other embodiments a single dose may administered via multiple applications of the composition to different points of the target tumor, or multiple doses of the adenovirus or adenoviral vector may be administered via repeated administrations of a particular dose. The number of administrations can be from about 2 to about 50 administrations or more (including all integers between 2 and 50) over a therapeutic period. The number of administrations will depend on the tumor location, tumor size, tumor type, and the like.

The disclosed method can be performed in combination with other therapeutic methods to achieve a desired biological effect in a patient. Ideally, the disclosed method may include, or be performed in conjunction with, one or more cancer treatments. The choice of cancer treatment used in combination with the disclosed method will depend on a variety of factors, including the cancer/tumor type, stage and/or grade of the tumor or cancer, the patient's age, etc. Suitable cancer treatments that may be employed include, but are not limited, surgery, chemotherapy, radiation therapy, immunotherapy, hormone therapy, and stem cell transplantation. In one embodiment, the disclosed method further comprises treating the tumor cells with a chemotherapeutic agent. Any suitable chemotherapeutic agent can be used in the disclosed method, including, for example, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, anti-vascular endothelial growth factor compounds (“anti-VEGFs”), anti-epidermal growth factor receptor compounds (“anti-EGFRs”), 5-fluorouracil, and the like. The type and number of chemotherapeutics used in the disclosed method will depend on the standard chemotherapeutic regimen for a particular tumor type.

In other embodiments, the disclosed method may further comprise treating the tumor or cancer cells with a cytokine, such as any of the cytokines disclosed herein. Ideally, the cytokine exhibits therapeutic efficacy against cancer (e.g., IL-2 and IFN-α) (see, e.g., Lee. S. and K. Margolin, Cancers (Basel), 3 (4): 3856-3893 (2011); and Ardolino et al., Oncotarget, 6 (23): 19346-19347 (2015)).

In another embodiment, the disclosed method may further comprise treating the tumor or cancer cells with an immune checkpoint regulator. Immune checkpoints are molecules on immune cells that must be activated or inhibited to stimulate immune system activity. Tumors can use such checkpoints to evade attacks by the immune system. The immune checkpoint regulator may be an antagonist of an inhibitory signal of an immune cell, also referred to as a “checkpoint inhibitor,” which blocks inhibitory checkpoints (i.e., molecules that normally inhibit immune responses). For example, the immune checkpoint regulator may be an antagonist of A2AR, BTLA, B7-H3, B7-H4, CTLA4, GAL9, IDO, KIR, LAG3, PD-1, TDO, TIGIT, TIM3 and/or VISTA. Checkpoint inhibitor therapy therefore can block inhibitory checkpoints, restoring immune system function. Currently approved checkpoint inhibitors target the molecules CTLA4, PD-1, and PD-L1, and include ipilimumab (YERVOY®), nivolumab (OPDIVO®), pembrolizumab (KEYTRUDA®), atezolizumab (TECENTRIQ®), avelumab (BAVENCIO®), and durvalumab (IMFINZI®). Any suitable checkpoint inhibitor, such as those described in, e.g., Kyi. C. and M. A. Postow, Immunotherapy, 8(7): 821-37 (2016); Collin, M., Expert Opin Ther Pat., 26(5): 555-64 (2016); Pardoll, D. M., Nat Rev Cancer, 12(4): 252-6 (2012); and Gubin et al., Nature, 515(7528): 577-81 (2014)) may be used in combination with the disclosed method. In other embodiments, the immune checkpoint regulator may be an agonist of an immune cell stimulatory receptor, such as an agonist of BAFFR, BCMA, CD27, CD28, CD40, CD122, CD137, CD226, CRTAM, GITR, HVEM, ICOS, DR3, LTBR, TACI and/or OX40. While an immune checkpoint regulator desirably is administered in a composition or formulation that is separate from a composition or formulation comprising the adenovirus or adenoviral vector, in some embodiments the adenovirus or adenoviral vector comprises a nucleic acid sequence encoding an immune checkpoint regulator. For example, the adenovirus or adenoviral vector may comprise a nucleic acid sequence encoding a PD-1 inhibitor, such as an anti-PD-1 antibody (e.g., Pembrolizumab (KEYTRUDA®), Nivolumab (OPDIVO®), or Cemiplimab (LIBTAYO®)).

The following examples further illustrate the invention but, of course, should not be construed as in any way limiting its scope.

### EXAMPLE 1

This example describes the construction and characterization of a serotype 5 adenovirus in accordance with the present disclosure.

Various vectors were generated and evaluated herein. The vectors generated herein arc referred to as “UIO-524” vectors. UI-524 vectors are human serotype 5 conditionally replicative adenovirus (CRAd) vectors (Δ15E1A, E1B-19K deleted or E1B-19K mutated. ΔE3. F5/3) containing the DNA sequences of a SPARC promoter modified to include three hypoxia response elements (HREs) and 12 nuclear factor kappa B (NFκB) responsive elements (κBREs). The HREs serve as regulators for low oxygen environments, while the κBREs serve as a trigger for inflammation. Hypoxia and inflammation are characteristic of tumor microenvironments. The SPARC promoter is highly expressed in cancer associated fibroblast and endothelial cells in close contact with the malignant cell compartment. The modified SPARC promoter drives a mutant E1A gene encoding a protein with defective retinoblastoma (Rb) binding as discussed above.

Most or all of the E3 gene region of UIO-524 vectors is deleted (ΔE3) and the serotype 5 fiber knob is replaced by the serotype 3 knob (F5/3). The UIO-524 vectors also express the immune modulatory genes CD40L and 4-1BBL under the control of the cancer- and stem cell-specific hTERT promoter operatively linked to a minimal human CMV promoter, which provides for tumor specific expression of cytokines.

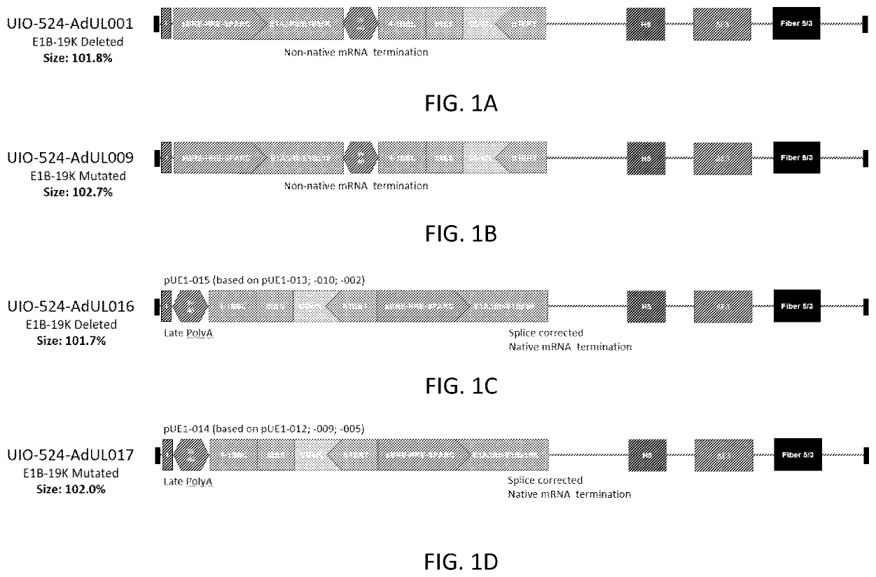

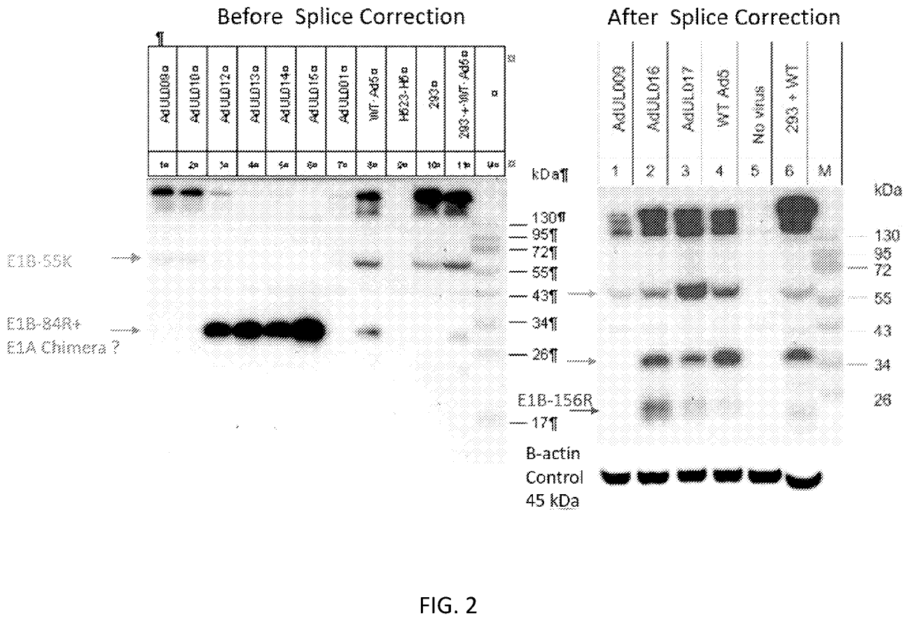

Various UIO-524 vectors having different configurations were generated and tested. These vectors are represented schematically in FIG. 1A-1D.

UIO-524-AdUL001 is shown in FIG. 1A. The vector comprises a nucleic acid sequence encoding a mutant E1A protein (e.g., a mutant E1A protein with defective Rb binding) under the modified SPARC promoter. The E1B-19K region is removed. The hTERT-CD40L/4-1BBL cassette is in reverse orientation compared to the original E1B transcriptional direction, and blocked by a dual directional SV40 polyA sequence to avoid interference with mutant E1A gene transcription.

UIO-524-AdUL009 is shown in FIG. 1B. The vector comprises a nucleic acid sequence encoding a mutant E1A protein (e.g., a mutant E1A protein with defective Rb binding) under the modified SPARC promoter. The E1B-19K region is mutated to be transcriptionally silenced. The hTERT-CD40L/4-1BBL cassette is inserted in reverse orientation compared to the original E1B transcriptional direction, and blocked by a dual directional SV40 polyA sequence as with UIO-524-AdUL001.

UIO-524-AdUL016 is shown in FIG. 1C. The vector comprises a nucleic acid sequence encoding a mutant E1A protein (e.g., a mutant E1A protein with defective Rb binding) under the modified SPARC promoter. The E1B-19K region is removed. The hTERT-CD40L/4-1BBL cassette is located between the left ITR and the E1 region of the adenovirus genome in reverse orientation compared to the E1A and E1B transcriptional direction. In contrast to the UIO-524-AdUL001 vector shown in FIG. 1A, the cassette transcription terminates with a late polyA sequence (e.g., SV40).

UIO-524-AdUL017 is shown in FIG. 1D. The vector comprises the same features as UIL-524-AdUL016, with the exception that the E1B-19K region is mutated to be transcriptionally silenced, rather than deleted.

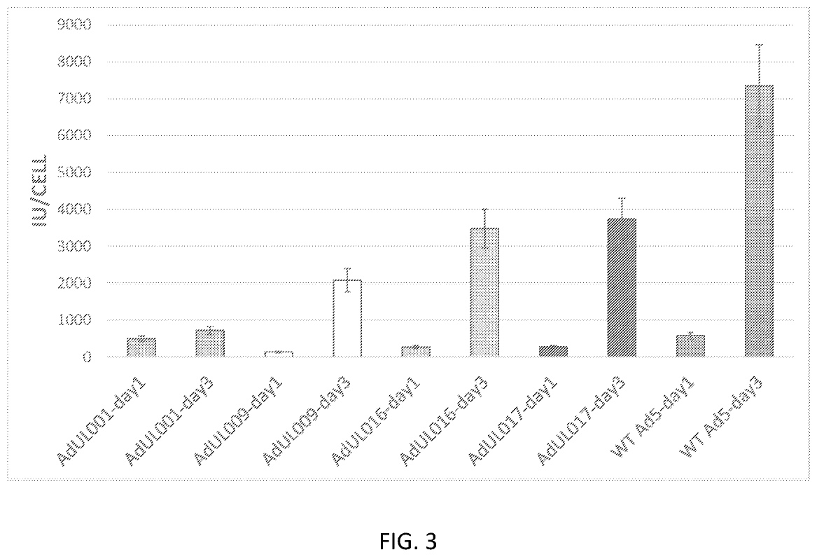

The vectors shown in FIG. 1A-1D along with other designed vectors were used to evaluate E1B-55K expression by western blotting. E1B-55K expression is essential for high levels of productivity in non-complimentary manufacturing cell lines like A549 or HeLa. Results are shown in FIG. 2. Results demonstrate that after splice correction E1B-55K expression is restored. For AdUL001 and AdUL009 the SD2 splice donor and SA3 acceptor was destroyed by an EcoRV site and SV40 pA. The E1B-55K transcript without a splice was inefficiently expressed. Early SV40 pA was used in the expression cassette. AdUL009-growing better than AdUL001, but neither virus accumulates in cells sufficiently for commercial scale production. For AdUL016 and AdUL017 the DS2 splice donor was corrected. Relocating the expression cassette there was no interference with the SV40 pA after E1B. Therefore, both E1B-55K and chimera were formed. Late SV40 pA is used in the expression cassette for improved termination of transcription. The amount of E1B-55K compared to WTAd5 for AdUL009 was 24%; for AdUL016 was 64%, and for AdUL017 was 186%.

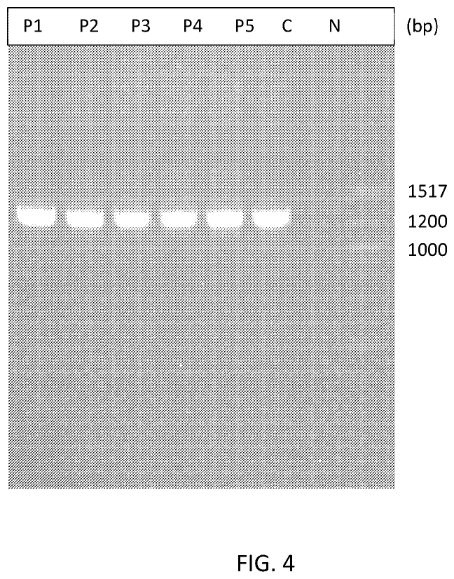

Viral replication was next evaluated. Crude viral stocks were generated by infecting A549 cells with AdUL001, AdUL009. AdUL016, AdUL017, and WTAd5. Crude cell lysates were tittered by hexon immunostaining. A549 cells were infected with each virus at MOI=3, in duplicate, and harvested at days 1 and 3 post-infection. Samples were titered by hexon immuno-staining. Results of replication of AdUL001, AdUL009, AdUL016 and AdUL017 in A549 cells are shown in FIG. 3. Replication for AdUL001 was ˜10%, for AdUL009 was about ˜25%, and for AdUL016 & AdUL017 was about ˜50% of WTAd. The average CsCl vector production indicated a particle (vp)/Infectious Unit (IU)=8 (n=4). Consequently, the burst size (defined as particle (vp)/cell) is approximately 70K vp for WTAd5, 35K vp for AdUL016 & AdUL017, 20K vp for AdUL009, and 7K vp for AdUL001. The maximum burst size expected for WTAd5 is about 100K vp/cell. These data indicates that the backbone of AdUL016 & AdUL017 were successfully corrected, and these adenovirus vectors are able to produce large amount of virus progeny that is commercially feasible.

Genomic stability of UIO-524 vectors was next assessed. Stability was assessed over five passages. Specifically, a PCR assay was performed to assess the integrity of the SPARC promoter region in the viral DNA extracted from infected A549 cells (passages 1-5). For templates, viral DNA was purified from the cell pellets using the Hirt method.

Hirt AdUL009 DNAs from passage P1 thru P5 and pAdUL009 cosmid DNA were assayed at dilution 10x. For AdUL009 P1-P5 Hirt DNAs, all were diluted 10χ in H2O. For pAdUL009, cosmid DNA 0.5 μg/μL was diluted 10× in H2O. Primers for SPARC detection were as follows:

249A: 5′-GGCGGGTGACGTAGTAGTGT-3 (SEQ ID NO: 13) located at 95-114 in AdUL009 genome.

245G: 5′-CTGGCGGCCATTTCTTCGGTAATAACACCT-3 (SEQ ID NO: 14) located at 1360C-1389C in AdUL009 genome.

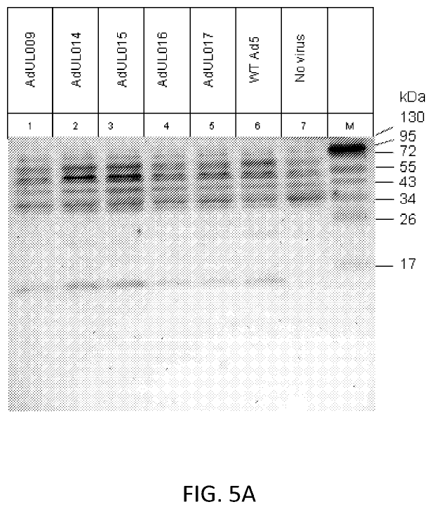

Following amplification, PCR products were assessed by gel electrophoresis using a 0.8% standard agarose gel and 2 μL PCR reaction/lane. Results are shown in FIG. 4. Lanes P1-P5 show AdUL009 Hirt DNA for passages 1-5, respectively. Lane C shows pAdUL009 cosmid DNA. Lane N shows negative control for PCR (H2O). Lane M shows 1 kb DNA ladder (NEB #3232). Expected fragment size is 1295 bp. Results demonstrate that there were no changes in the specific band. Accordingly, the virus AdUL009 is stable.

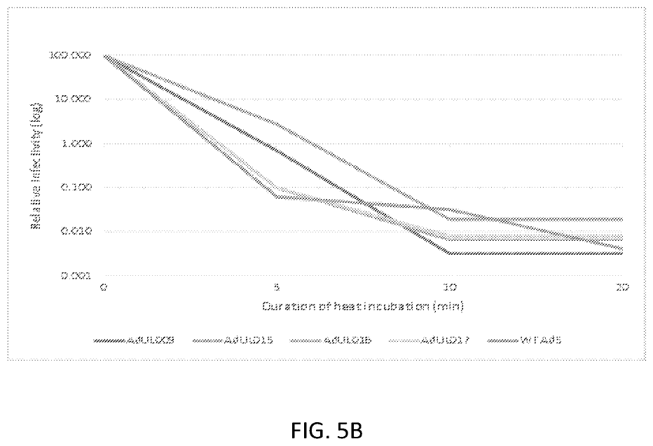

Heat stability of UIO-524 vectors was next evaluated. Samples of adenovirus particles were diluted 1:5 in DMEM/2% FCS, 0.5% glycerol and incubated at 48° C. for various periods of time. Residual infectivity was determined by immuno-histochemistry using an Adeno-X Rapid Titer kit (Takara #632250) assay on HEK293 cells and is expressed as a proportion of initial infectivity, plotted on a logarithmic scale. Results are shown in FIG. 5A-5B. FIG. 5A shows a western blot demonstrating that the pIX protein is expressed correctly in all vectors, regardless of splice SD2 deletion. The 30 kDa band (or 26 kDa on a 15% gel) is not an E1B/pIX chimera. FIG. 5B is a graph showing the relative infectivity as a function of duration of incubation (in minutes). The heat stability of the tested vectors (AdUL009, AdUL015. AdUL016, AdUL017) is essentially not different from stability of WTAd5. AdUL015 appear to be more stable than WTAd5, but this lot was the only one not CsCl purified. AdUL015 is identical to AdUL016, but using the early SV40 polyA sequence instead of the late polyA sequence of SV40. Accordingly, overexpression of the 30 kDa chimeric protein (shown in FIG. 2) is not causing heat instability.

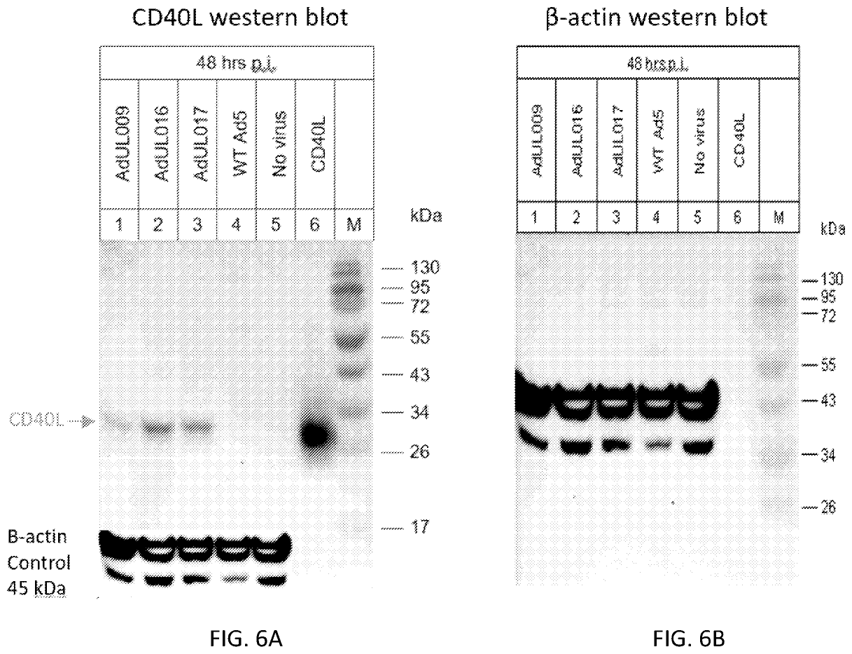

The ability of UIO-524 vectors to infect cells and produce the desired gene products was next evaluated. To evaluate the ability of the vectors to produce CD40L, A549 cells were infected with AdUL009, AdUL016, AdUL017, WTAd5 virus. Cell lysates were obtained and expression of CD40L and β-actin was evaluated. Results are shown in FIG. 6A (CD40L) and FIG. 6B (β-actin). For CD40L, Polyclonal anti-CD40L antibody R&D #AF617 was used. For β-actin. Polyclonal anti-β-actin antibody R&D #MAB8929 (45 kDa) was used. Results demonstrate that the amount of CD40L produced is more substantially in AdUL016 (100%) and AdUL017 (90%) than in AdUL009 (21%). CD40L lane shows the purified peptide control (expected MW 22-26 kDA).

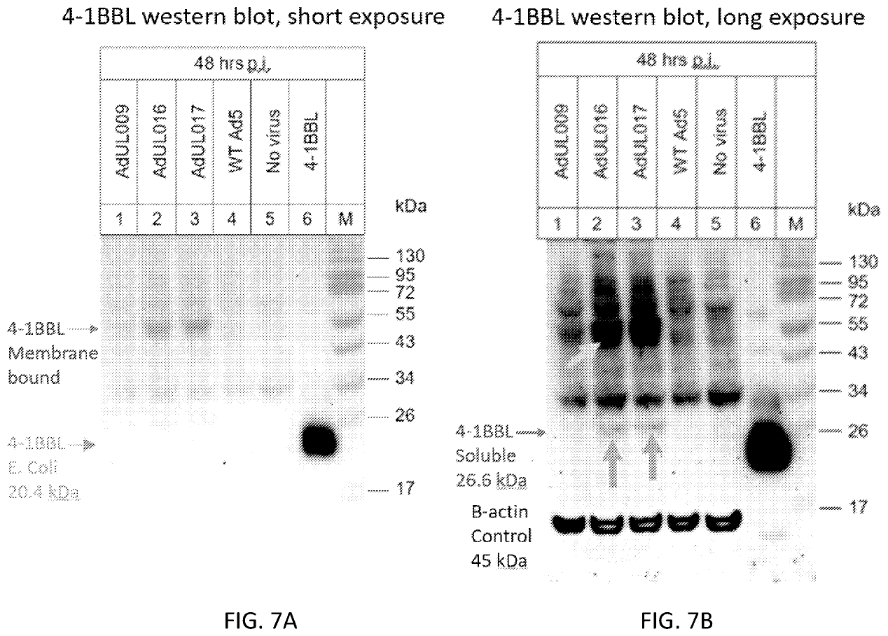

To evaluate the ability of UIO-524 vectors to infect cells and produce 4-1BBL, RT4 cells were selected to avoid high 4-1BBL background otherwise seen in A549 cells. RT4 cells were infected with adenoviral vectors AdUL009, AdUL016, AdUL017, WtAd5. Cell lysates were obtained and expression of 4-1BBL was evaluated by western blot. Results are shown in FIG. 7A (short exposure time during imaging) and FIG. 7B (long exposure time during imaging). For 4-1BBL, Monoclonal anti-4-1BBL antibody from Novus Biologicals #MAB2295 was used. In the blots, the large 50 kDa band is the membrane bound glycosylated form of 4-1BBL, and the 26.6 kDa band is the soluble form monomer. 4-1BBL lane shows the purified peptide control (expected MW 20.4 kDA). Results demonstrate that the amount of 4-1BBL produced is substantially more in AdUL016 (100%) and AdUL017 (94%) then in AdUL009 (16%).

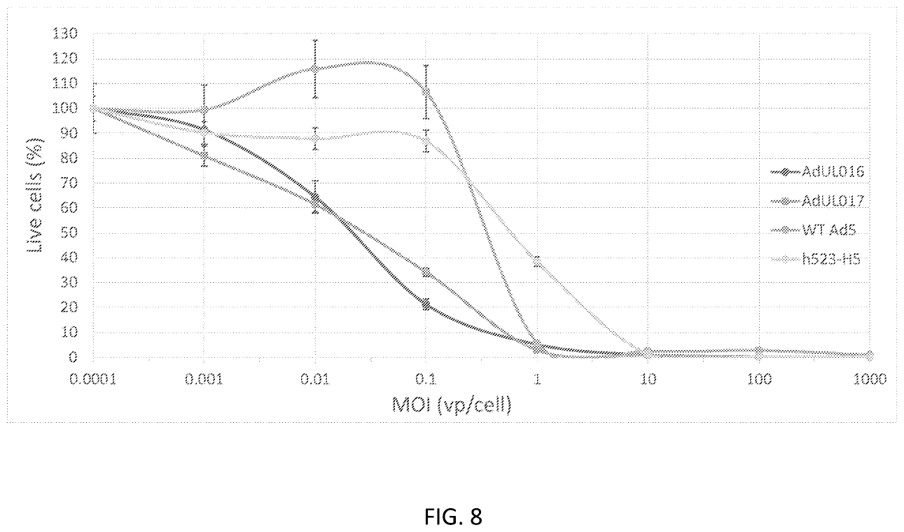

In vitro lytic activity of UIO-524 vectors was next evaluated. A549 cells were infected with a dilution series of AdUL016, AdUL017, WTAd5, or h523-H5. After 6 days, an MTS cytotoxicity assay was performed (Promega, Inc.). Results are shown in FIG. 8. The viability of each infected culture is expressed as a percentage of uninfected controls. Each data point is the mean of two replicate. Approximate 50% Effective dose (ED₅₀) is calculated from the graphs. AdUL016, and AdUL017 have identical arming cassettes driven by CMV/hTERT promoter and replicate reasonably well in A549 cells. The observed ED50 is ˜0.02 indicating that these viruses kill cells 25× better than WTAd. These viruses are mutated in the E1B-19K protein, and therefore cells are not protected from apoptosis. H523-H5 has the same cassette but is driven with only the hTERT promoter and does not replicate well in A549 cells. Its observed ED50 is ˜0.7, which is very close to WTAd. WTAd replicates well but does not have an expression the cassette. The WTAd has an expected ED50 of ˜0.5. At higher MOI WTAd is more effective than H523-H5. Taken together, without wishing to be bound by theory, these results indicate that viral replication (and/or E1A) kills cells, but one of the proteins in the cassette (probably 4-1BBL) might be a contributing lytic factor as well. The deletion of E1B-19K protein might be the most significant contributing factor.

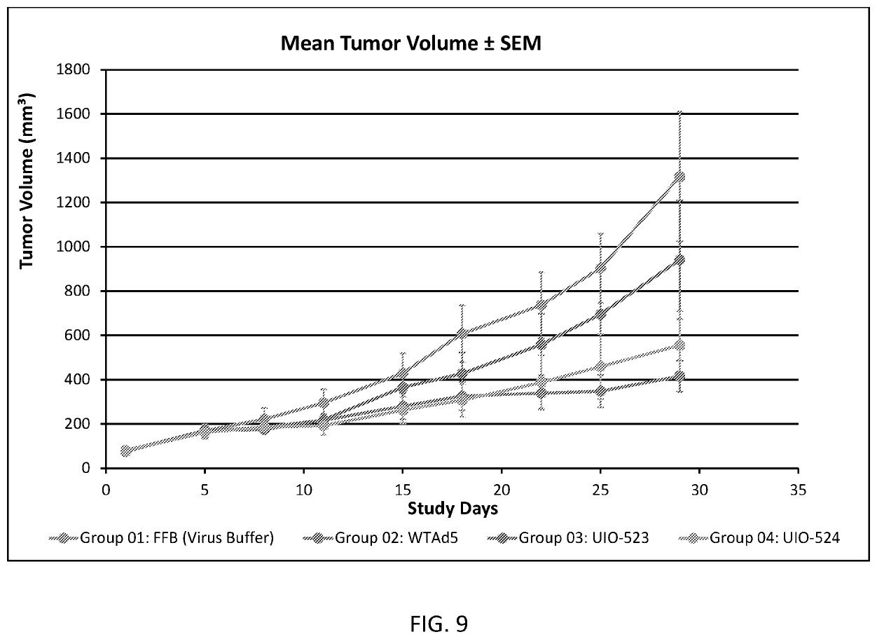

An in vivo efficacy study of tumor killing activity of AdUL016 was next performed. A cell line-derived xenograft (CDX) mouse model was used. Female NOD/SCID mice were obtained from Charles River and the experiment performed by Crown Bioscience San Diego. Mice were injected subcutaneously with 5×10e6 A549 cells and tumor growth was monitored. When tumors reached 80-100 mm³, mice were subsequently injected with 1.25×10e10 vp per dose in a total volume of 50 microliters on days 1, 3, and 5. Four study groups were used, with 8 mice per group. Groups were: Group 1—formulation buffer (FFB), Group 2—WTAd. Group 3—h523-H5 (also referred to as “UIO-523”), Group 4—AdUL016 (also referred to as “UIO-524”). Body weights and tumor volume was measured for 4 weeks, and tumor growth inhibition T/C ratio was calculated. T/C ratio results were: WTAd: 0.32, UIO-523:0.72, UIO-524:0.42, indicating that in vivo UIO-524 has a much better tumor killing effect than UIO-523.Results are shown in FIG. 9.

Results from the above-described experiments are summarized in Table 1.

TABLE 1

| | | | | | | A549 | In Vivo | | |
|---|---|---|---|---|---|---|---|---|---|
| | Genomic | | E1B-55K | CD40L | 4-1BBL | Killing | Efficacy | Heat | Total |
| Vector | Stability | Growth | Expression | Expression | Expression | (ED50) | (T/C ratio) | Stability | Score |
| AdUL009 | 100% | 28% | 24% | 21% | 16% | 0.02 | ND | 100% | ++ |
| AdUL016 | ND | 47% | 64% | 100% | 100% | 0.02 | 0.02 | 100% | ++++ |
| AdUL017 | ND | 51% | 186% | 90% | 94% | 0.02 | 0.02 | 100% | ++++ |
| WTAd5 | 100% | 100% | 100% | 0% | 0% | 0.5 | 0.5 | 100% | NA |

Stability: Relative PCR band strength after 5 passages compared to passage 1. AdUL016 & AdUL017 is expected to be stable as each has the same κBRE repeats and hTERT promoter as AdUL009.

Growth: Relative virus growth at 3 days' time point compared to WTAd5 defined as 100%.

Expression: E1B-55K expression compared to WTAd5 defined as 100%; corrected by gel loading. CD40L and 4-1BBL expression compared to the strongest band defined as 100%, corrected by gel loading.

Cell Viability (Killing): Viral particle (vp) moi at ED50. Lower number indicates more lytic activity.

It has been identified in certain cases that the anti-tumor immune response is much more important for clearing tumors than is the direct oncolytic effect of the virus. Therefore, better killing does not necessarily identify a better virus.

The results in Table 1 were used to assign a score to each vector. This score is shown in the Total Score column. Genomic stability is ignored for time being.

Conclusion: AdUL016 and AdUL017 achieved a much higher score compared to AdUL009. Given that AdUL016 genome size (101.7%) is smaller than AdUL017 (102.0%), it might be preferable to be used in future constructs as starting backbone.

### EXAMPLE 2

IL-21 was identified as a potential cytokine to incorporate into the vectors described herein. Human IL-21 is a 15.4 kDa protein consisting of 132 amino acid residues and cross reacts with monkey and mouse. IL-21 is a pleiotropic cytokine produced by CD4+ T cells in response to antigenic stimulation. Biological effects of IL-21 include inducing differentiation of T cell-stimulated B cells into plasma cells and memory B cells; stimulation of IgG production in conjunction with IL-4; inhibition of the generation of Treg cells etc. Moreover, IL-21 promotes the anti-tumor activity of CD8+ T cells and NK cells. The IL-21/IL-21R interaction triggers a cascade of events, which includes activation of the tyrosine kinases JAK1 and JAK3, followed by activation of the transcription factors STAT1 and STAT3.

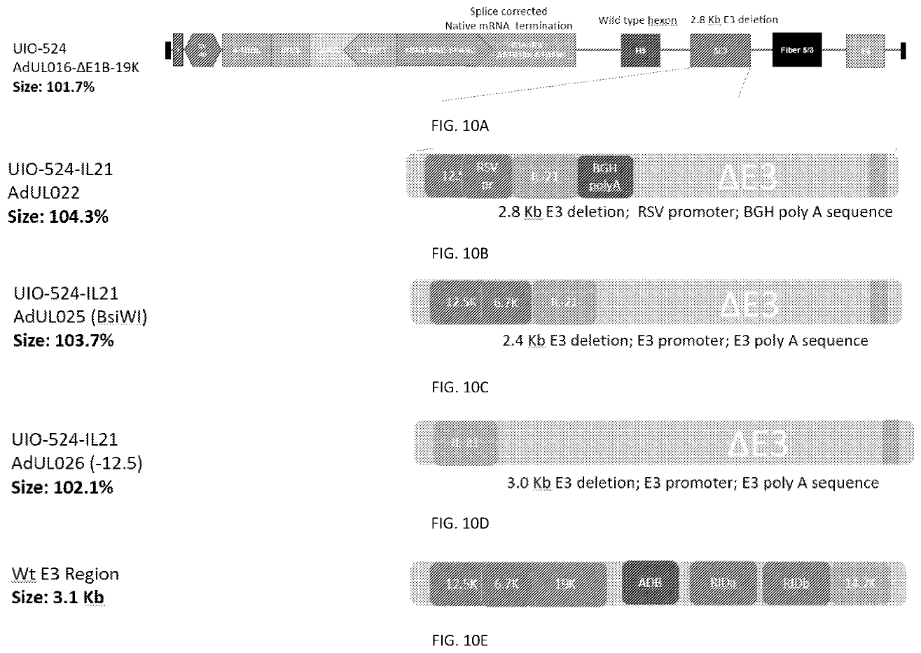

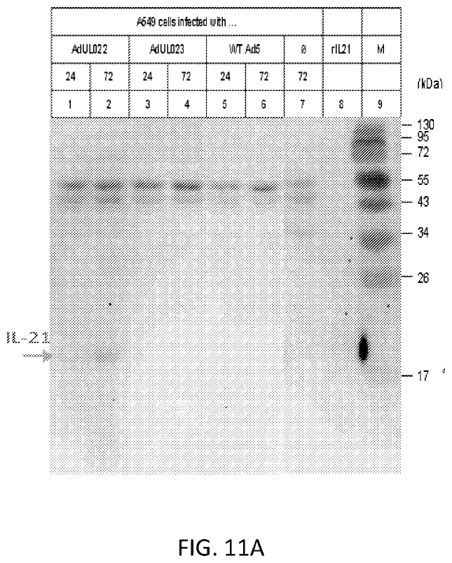

Multiple vectors incorporating IL-21 were designed and generated in the AdUL016 background. Exemplary vectors are shown in FIG. 10B-10D. For reference. AdUL016 (containing a 2.8 Kb deletion in the E3 region) is shown in FIG. 10A. All vectors shown in FIG. 10B-10D contain the nucleic acid sequence encoding IL-21 under control of the respective promoter in the deleted E3 region. The wt E3 region is shown in FIG. 10E.

FIG. 10B shows the UIO-524-IL21 vector AdUL022. AdUL022 comprises a 2.8 Kb deletion in the E3 region of the adenoviral genome. AdUL022 comprises a nucleic acid sequence encoding IL-21 under control of an RSV promoter, and a BGH polyA sequence.

FIG. 10C shows the UIO-524-IL21 vector AdUL025. AdUL025 comprises a 2.4 Kb deletion in the E3 region of the adenoviral genome. AdUL025 comprises a nucleic acid sequence encoding IL-21 under control of the adenovirus native E3 promoter, and an E3 polyA sequence.

FIG. 10D shows the UIO-524-IL21 vector AdUL026. AdUL026 comprises a 3.0 Kb deletion in the E3 region of the adenoviral genome. AdUL026 comprises a nucleic acid sequence encoding IL-21 under control of the adenovirus native E3 promoter, and an E3 polyA sequence.

UIO-524-IL21 vectors were tested for their ability to infect cells and induce expression of IL-21. A549 cells were infected with AdUL022, AdUL025, AdUL026, WTAd5.

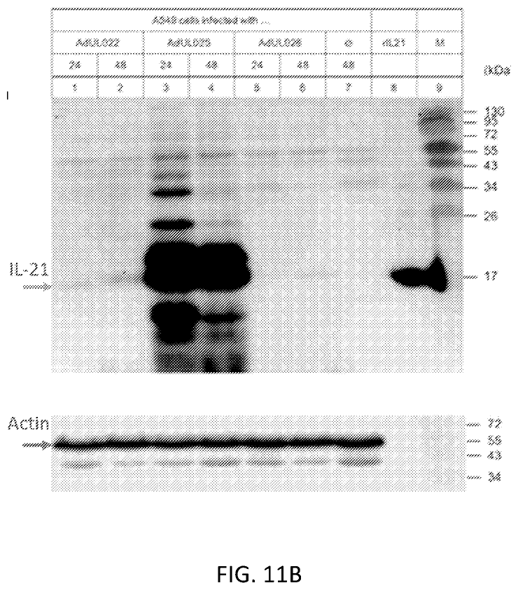

Cell lysates were isolated 24 hours and 48 hours, or 72 hours after infection and IL-21 expression was evaluated by western blot on 15% SDS-Page gels under reducing conditions. Lanes were loaded with 3 micrograms protein per lane. Antibodies used were goat polyclonal anti-hIL-21 antibody (R&D Cat #AF15001-100, 1:200), and Rabbit anti-goat IgG (HRP-conjugated. R&DHAF017, 1:1000). Signal was evaluated using SuperSignal West Pico PLUS chemiluminescent substrate (Thermo #34580) with an exposure time of 20×1 second. Results are shown in FIG. 11A and FIG. 11B. β-actin was used as a control, with a MW=45 KDa. Control lanes were included as no virus infection and recombinant IL-21 (rIL21, R&D #8879-IL-010/CF. 3.5 ng/lane, 17 kDa). The predicted MW of hL21=18.7 kDa.

The results show that small amounts of IL-21 are produced from unspliced mRNA in AdUL022 & AdUL026. Unexpectedly, exceptional large amounts of IL-21 were produced from the early spliced mRNA in AdUL025. The upper band in AdUL25 is unprocessed protein.

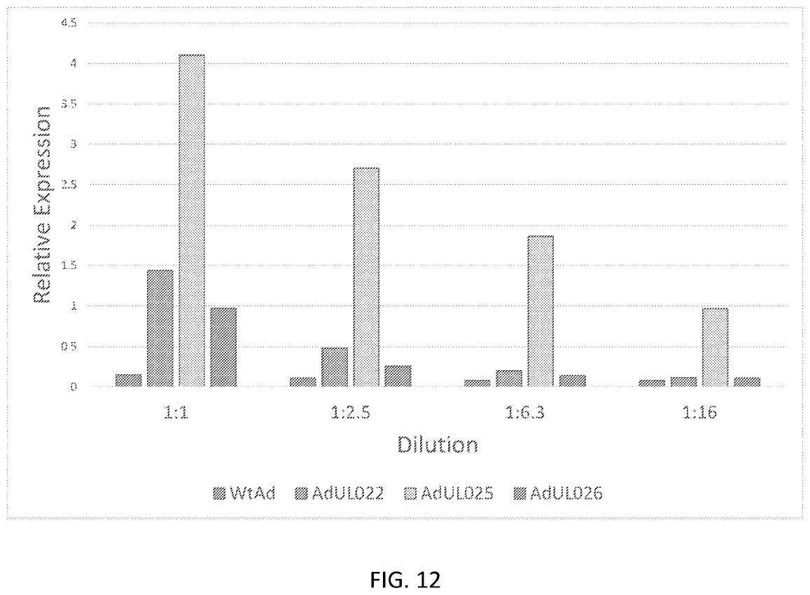

Expression of IL-21 was further evaluated by ELISA. Crude virus stocks were generated by infecting A549 cells with AdUL022, AdUL025, AdUL026, or WTAd5. Crude lysates were tittered by hexon immunostaining. A549 cells were infected with each virus at MOI=3, in duplicate. Cells were harvested at day 1 and samples were tittered by hexon immune staining. ELISA kit used (RayBiotech #ELH-IL21), following the manufacturer's protocol. Results were read on a Microplate Reader: Tecan i-control, 1.12.4.0 with an absorbance wavelength of 450 nm and a bandwidth of 10 nm. Results are shown in FIG. 12. Results of ELISA from cell supernatant comports with the western blot results from cellular proteins and show that AdUL022 and AdUL026 produce IL-21, and AdUL25 produces very large amounts of IL-21.

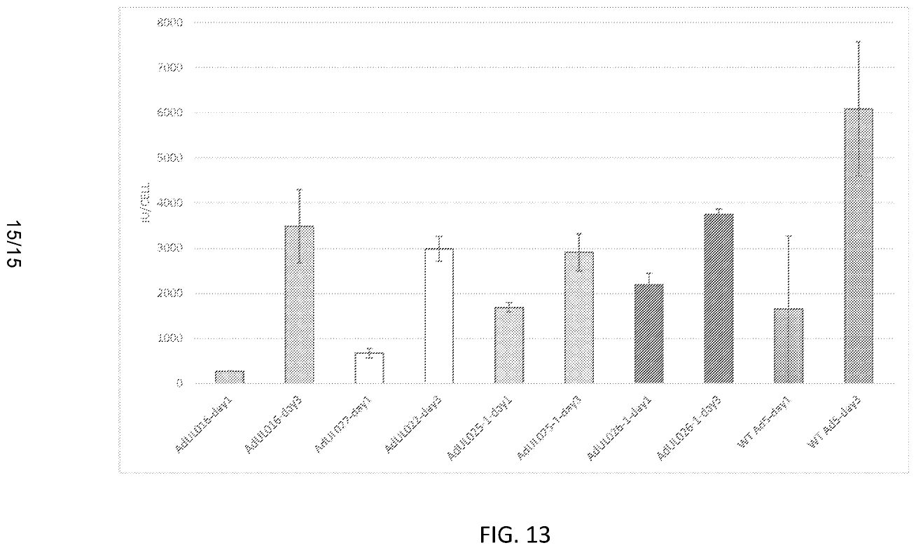

Replication of AdUL024-IL21 vectors was next assessed. Crude virus stocks were generated by infecting A549 cells with AdUL022, AdUL025, AdUL026, or WTAd5. Crude lysates were tittered by hexon immuno-staining. A549 cells were infected with each virus at MOI=3, in duplicate. Infected cells were harvested at days 1 and 3 post infection. Samples were tittered by hexon immuno-staining. The average CsCl vector production indicated a particle (vp)/Infectious Unit (IU)=8 (n=4). Consequently, the burst size (defined as particle (vp)/cell) is approximately 60K vp (WTAd5), 30K vp (AdUL026 & AdUL027), and 15K vp (AdUL022). The maximum burst size expected for WTAd5 is about 100K vp/cell. Results are shown in FIG. 13. Replication of AdUL016, AdUL022, AdUL025, and AdUL026 was about 50% of WTAd which would allow commercially feasible production of these adenovirus vectors.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising.” “having.” “including.” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A serotype 5 adenovirus or adenoviral vector comprising:
(a) a nucleic acid sequence encoding a mutant E1A protein operatively linked to a first promoter that is responsive to hypoxia and inflammation,
(b) a deletion of all or part of the E1B-19K region of the adenoviral genome with the E1B-55K region intact,
(c) an expression cassette located between the left ITR and the E1 region of the adenovirus genome,
(d) one or more non-native nucleic acid sequences, each of which encodes an immune modulator and is operatively linked to a second promoter that is active in tumor cells,
(e) one or more non-native nucleic acid sequences, each of which encodes an immune modulator and is operatively linked to the adenovirus native E3 promoter, and
(f) a fiber protein comprising a serotype 3 adenovirus fiber knob domain.

2. The adenovirus or adenoviral vector of claim 1, which comprises a deletion of all or part of the E3 region of the adenoviral genome.

3. The adenovirus or adenoviral vector of claim 2, wherein the one or more non-native nucleic acid sequences are located in the deleted E3 region of the adenoviral genome.

4. The adenovirus or adenoviral vector of claim 2, wherein the one or more non-native nucleic acid sequences are located in expression cassette located between the left ITR and the E1 region of the adenovirus genome.

5. The adenovirus or adenoviral vector of any one of claims 1-4, wherein the nucleic acid sequence encoding a mutant E1A protein comprises a deletion of 15 nucleotides within a retinoblastoma (Rb) protein binding region of the E1A protein.

6. The adenovirus or adenoviral vector of any one of claims 1-5, wherein the first promoter is a secreted protein acidic and rich in cysteine (SPARC) promoter which comprises one or more hypoxia-responsive elements (HREs) and one or more nuclear factor kappa B (NF-κB) inflammation responsive elements (κBRE).

7. The adenovirus or adenoviral vector of any one of claims 1-6, comprising an exogenous nucleic acid sequence encoding CD40 ligand (CD40L) and an exogenous nucleic acid sequence encoding 4-1BB ligand (4-1BBL).

8. The adenovirus or adenoviral vector of any one of claims 1-6, comprising an exogenous nucleic acid sequence encoding IL-21.

9. The adenovirus or adenoviral vector of any one of claims 1-8, wherein the second promoter is a composite human telomerase reverse transcriptase (hTERT) promoter operatively linked to a minimal human CMV promoter.

10. A serotype 5 adenovirus or adenoviral vector comprising:
(a) a nucleic acid sequence encoding a mutant E1A protein operatively linked to a secreted protein acidic and rich in cysteine (SPARC) promoter which comprises one or more hypoxia-responsive elements (HREs) and one or more nuclear factor kappa B (NF-κB) inflammation responsive elements (κBRE),
(b) a deletion of all or part of the E1B-19K region of the adenoviral genome with the E1B-55K region intact,
(c) a first exogenous nucleic acid sequence encoding CD40 ligand (CD40L) and a second exogenous nucleic acid sequence encoding 4-1BB ligand (4-1BBL), wherein the first and second exogenous nucleic acid sequences are (i) separated by an internal ribosome entry site (IRES) and (ii) operatively linked to a composite human telomerase reverse transcriptase (hTERT) promoter operatively linked to a minimal human CMV promoter and (iii) transcribed from an expression cassette located between the left ITR and the E1 region of the adenovirus genome,
(d) a third exogenous nucleic acid sequence encoding IL-21 operatively linked to the adenovirus native E3 promoter, and
(e) a fiber protein comprising a serotype 3 adenovirus fiber knob domain.

11. A composition comprising the adenovirus or adenoviral vector of any one of claims 1-10 and a pharmaceutically acceptable carrier.

12. The composition of claim 11, which comprises about 1×10⁸ to about 1×10¹⁴ viral particles (vp) of the adenovirus or adenoviral vector

13. The composition of claim 12, which comprises about 1×10¹⁰ to about 1×10¹² vp of the adenovirus or adenoviral vector

14. A method of inducing cytotoxicity in tumor cells which comprises contacting tumor cells with the composition of claim 11, whereupon the E1B-55K protein and the exogenous nucleic acid sequences are expressed in the tumor cells and cytotoxicity is induced.

15. The method of claim 14, wherein the tumor cells are in vitro.

16. The method of claim 14, wherein the tumor cells are in vivo.

17. The method of claim 16, wherein the tumor cells are in a mammal

18. The method of claim 17, wherein the mammal is a human.

19. The method of any one of claims 14-18, wherein the tumor cells are ovarian cells.

20. The method of any one of claims 14-19, further comprising treating the tumor cells with a chemotherapeutic agent.

21. The method of any one of claims 14-20, further comprising treating the tumor cells with a cytokine or checkpoint inhibitor (CI).


SEQUENCE LISTING (14 sequences)

SEQ ID NO: 1 - DNA, 993 bp, synthetic construct
  source 1..993: mol_type = other DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaa

SEQ ID NO: 2 - DNA, 978 bp, synthetic construct
  source 1..978: mol_type = other DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaa

SEQ ID NO: 3 - protein, 5 aa, synthetic construct
CRGDC

SEQ ID NO: 4 - protein, 9 aa, synthetic construct
CXCRGDCXC

SEQ ID NO: 5 - protein, 9 aa, synthetic construct
CDCRGDCFC

SEQ ID NO: 6-7 - skipped (2 entries)

SEQ ID NO: 8 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 9 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 10 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 11 - protein, 129 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 12 - protein, 132 aa, synthetic construct
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - DNA, 20 bp, synthetic construct
  source 1..20: mol_type = other DNA
ggcgggtgacgtagtagtgt

SEQ ID NO: 14 - DNA, 30 bp, synthetic construct
  source 1..30: mol_type = other DNA
ctggcggccatttcttcggtaataacacct